(19) United States
(12) Patent Application Publication
(10) Pub. No.: US 2023/0405653 A1
(43) Pub. Date: Dec. 21, 2023

(54) METHOD FOR SANITIZING WASTE

(71) Applicant: Renescience A/S, Fredericia (DK)

(72) Inventors: Hanne Risbjerg SØRENSEN, Fredericia (DK); Steen Gustav STAHLHUT, Fredericia (DK)

(73) Assignee: Renescience A/S, Fredericia (DK)

(21) Appl. No.: 18/035,247

(22) PCT Filed: Nov. 3, 2021
(86) PCT No.: PCT/EP2021/080529

§ 371 Date: May 3, 2023

(30) Foreign Application Priority Data
Nov. 4, 2020 (EP) 20205654.5
Nov. 16, 2020 (EP) 20207700.4
Dec. 2, 2020 (EP) 20211202.5

(51) Int. Cl. B09B 3/60; A61L 11/00

(52) U.S. Cl. CPC B09B 3/60; B09B 2101/70; A61L 11/00

(57) ABSTRACT

The present invention relates to a method for sanitizing waste, where waste having specific bacterial counts are subjected to an enzyme composition at a pH between 30 and 6.0 and at a temperature of between 40° C. and 60° C., the liquid is separated and the waste is subjected to the enzyme composition for a period of 10 to 30 hours to obtain at least partial reduction in bacterial count.

### FIELD OF THE INVENTION

The present invention relates to a method for sanitizing waste, the sanitized waste and bioliquid being produced from the method and biogas being produced from the bioliquid.

### BACKGROUND

There is a great interest to employ methods in which the energy stored within waste comprising organic material is utilized to the fullest. Agricultural waste, household waste and municipal waste are examples of waste containing a high content of dry matter and a certain content of organic material, which is biodegradable. Considerable interest has arisen in development of efficient and environmentally friendly methods of processing such waste, to maximize recovery of their inherent energy potential (the bio-degradable material) and recovery of recyclable materials. One significant challenge in “waste to energy” processing has been the heterogeneous nature of waste, such as municipal solid waste (MSW).

The commonly used methods for treatment and subsequent disposal of waste such as household, agricultural or municipal waste include among others incineration, landfill, and composting, where the method of choice often depends on e.g. the content of organic material compared to the content of non-organic material. However, these methods do not directly provide an optimum utilization of the energy stored within the organic material.

Pre-sorting of household waste may sometimes be provided by the consumers or by the waste station and this reduces the pollution released by e.g. incineration and simplifies the degradation of the organic waste into valuable end-products. However, pre-sorting may not be efficient in separating all non-biodegradable material such as metal and glass from the organic waste.

In methods where the organic contents of the waste are liquefied and/or saccharified, while the non-organic contents are maintained in their solid phase, followed by separation of the solid and the liquid phases, pre-sorting simplifies the process, but is not a necessity.

An example of an environmentally friendly waste processing method is the biologically based method applied by Renescience, wherein waste comprising organic matter, such as ordinary unsorted and/or sorted/partially sorted household waste, is mixed with water, enzymes and/or microorganisms in order to liquefy and/or saccharify organic waste such as food waste, cardboard, paper, labels and similar. Such method is described in international patent application WO 2013/185778, which describes methods and compositions for biomethane production from MSW. MSW, which may be unsorted, is concurrently treated with enzyme and a bacterial culture to release the energy saved in the biodegradable material in MSW and turn it into a bioliquid that can be used for production of biogas via an anaerobic digestion process.

Anaerobic digestion (AD) may deactivate viable pathogens, including parasite, virus, and the pathogens harbouring antibiotic resistance genes. The review article “Is anaerobic digestion a reliable barrier for deactivation of pathogens in bio-sludge? Elsevier, Vol. 668, Pages 893-902, Jun. 10, 2019” aims to provide a critical overview regarding the deactivation of sludge-associated pathogens by AD, through which a serious concern on the effectiveness and rationality of AD towards sludge pathogens control was raised. Meanwhile, the underlying deactivation mechanisms and affecting factors are discussed, with the focus on pathogen-associated modelling, engineering design and technological aspects of AD.

It was previously believed that waste fractions should be hygienized for example by pre-treatment at temperatures of 90-95° C. before being used for producing a bioliquid. The effect of the pre-treatment is a sterilization/hygienization of the waste fraction, whereby undesired microorganism, e.g. pathogenic bacteria, were killed.

WO2013/185778 teaches that pre-heating of waste is not always necessary. The application shows that by addition of microorganisms (inoculation of EC12B) and enzymes to waste and allowing concurrent enzymatic treatment and microbial fermentation at temperatures of 45-75° C. for a time period of 212 hours or more, a safe fermentation can be achieved for at least some pathogenic bacteria.

However, it would be beneficial to treat MSW enzymatically and/or microbially in a safe, environmentally and economic way without prior pre-heating or at least by a method that requires less energy input for instance for increasing the temperature.

With the present invention, it has surprisingly been found a large part of the total amount of Enterobacteria species found in MSW can be significantly reduced, particularly one of the most common pathogenic bacterial in MSW, E. coli, by a method wherein the enzymatic and/or microbial treatment is carried out in a bioreactor at a pH between 3.0 and 6.0 and at a temperature of between 40° C. and 60° C. for a period of only 10 to 30 hours. This invention is accordingly particularly beneficial in applying lower temperatures and shorter duration of enzymatic and/or microbial treatment than previously believed to be necessary.

### SUMMARY OF THE INVENTION

The present invention pertains to a method for sanitizing waste, the method comprising:
a) Subjecting waste comprising biodegradable material and non-biodegradable material and having a total bacterial count of at least 2.5×10⁸ CFU/gram waste, a bacterial count of E. coli of at least 1.5×10⁶ CFU/gram waste or a bacterial count of Enterobacteriaceae of at least 1.5×10⁸ CFU/gram waste, to enzymatic and/or microbial treatment in a bioreactor at a pH between 3.0 and 6.0 and at a temperature of between 40° C. and 60° C. for a period of to 30 hours to obtain at least partial reduction in bacterial count.

The method may further comprise:
b) subjecting the treated waste from step a) to one or more separation step(s), whereby a bioliquid and a solid fraction is provided;
c) subjecting said bioliquid and/or solid fraction to downstream processing

The invention further relates to a bioliquid and non-biodegradable material obtainable by the process of the invention.

The method of the current invention is advantageous as it sanitizes waste at low temperatures in a safe and economical way.

### Definitions

“Biodegradable matter” refers to organic matter that can be partly or completely degraded into simple chemical compounds such as mono-, di- and/or oligosaccharides, amino acids and/or fatty acids by microorganisms and/or by enzymes. Biodegradable matter is generally organic material that provides a nutrient for microorganisms, such as mono-, poly- or oligosaccharides, fat and/or protein. These are so numerous and diverse that a huge range of compounds can be biodegraded, including hydrocarbons (oils), polycyclic aromatic hydrocarbons (PAHs), polychlorinated biphenyls (PCBs) and pharmaceutical substances. Microorganisms secrete biosurfactant, an extracellular surfactant, to enhance this process.

“Cellulose” is a homopolysaccharide composed entirely of D-glucose linked together by [beta]-1,4-glucosidic bonds and with a degree of polymerisation up to 10,000. The linear structure of cellulose enables the formation of both intra- and intermolecular hydrogen bonds, which results in the aggregation of cellulose chains into micro fibrils. Regions within the micro fibrils with high order are termed crystalline and less ordered regions are termed amorphous. The micro fibrils assemble into fibrils, which form the cellulose fibres.

“Cellulosic material” means any material containing cellulose. Cellulosic material includes agricultural residue, herbaceous material (including energy crops), municipal solid waste, pulp and paper mill residue, wastepaper, textiles including cotton material and wood (such as forestry residue).

“Hemicellulose” is a complex heterogeneous polysaccharide composed of a number of monomer residues: D-glucose, D-galactose, D-mannose, D-xylose, L-arabinose, D-glucuronic acid and 4-O-methyl-D-glucuronic acid. Hemicellulose has a degree of polymerisation below 200, has side chains and may be acetylated. In softwood like fir, pine and spruce, galactoglucomannan and arabino-4-O-methyl-glucuronoxylan are the major hemicellulose fractions. In hardwood like birch, poplar, aspen or oak, 4-O-acetyl-4-methyl-glucuronoxylan and glucomannan are the main constituents of hemicellulose.

“Municipal solid waste” (MSW) refers to waste fractions which are typically available in a city, but that need not come from any municipality per se, i.e., MSW refers to every solid waste from any municipality but not necessarily being the typical household waste could be disposed from airports, universities, campus, canteens, general food waste, among others. MSW may be any combination of one or more of cellulosic, plant, animal, plastic, metal, or glass waste including, but not limited to, any one or more of the following: Garbage collected in normal municipal collections systems, optionally processed in a central sorting, shredding or pulping device, such as e.g., a Dewaster® or a reCulture®; solid waste sorted from households, including both organic fractions and paper rich fractions; Generally, municipal solid waste in the Western part of the world normally comprise one or more of: animal food waste, vegetable food waste, newsprints, magazines, advertisements, books and phonebooks, office paper, other clean paper, paper and carton containers, other cardboard, milk cartons and alike, juice cartons and other carton with alu-foil, kitchen tissues, other dirty paper, other dirty cardboard, soft plastic, plastic bottles, other hard plastic, non-recyclable plastic, yard waste, flowers etc., animals and excrements, diapers and tampons, cottonsticks etc., other cotton etc., wood, textiles, shoes, leather, rubber etc., office articles, empty chemical bottles, plastic products, cigarette buts, other combustibles, vacuum cleaner bags, clear glass, green glass, brown glass, other glass, aluminium containers, alu-trays, alu-foil (including tealight candle foil), metal containers (-Al), metal foil (-Al), other sorts of metal, soil, rocks, stones and gravel, ceramics, cat litter, batteries (botton cells, alkali, thermometers etc.), other non-combustibles and fines.

An oligosaccharide is a saccharide polymer containing a small number (typically three to ten) of monosaccharides. They are normally present as glycans: oligosaccharide chains linked to lipids or to compatible amino acid side chains in proteins, by N- or O-glygosidic bonds. N-linked oligosaccharides are always pentasaccharides attached to asparagine via a beta linkage to the amine nitrogen of the side chain. Alternately, O-linked oligosaccharides are generally attached to threonine or serine on the alcohol group of the side chain. Not all-natural oligosaccharides occur as components of glycoproteins or glycolipids. Some, such as the raffinose series, occur as storage or transport carbohydrates in plants. Others, such as maltodextrins or cellodextrins, result from the microbial breakdown of larger polysaccharides such as starch or cellulose.

“Organic” refers to materials that comprises carbon and are bio-degradable and include matter derived from living organisms. Organic material can be degraded aerobically (with oxygen) or anaerobically (without oxygen). Decomposition of biodegradable material may include both biological and abiotic steps.

Polysaccharides are polymeric carbohydrate molecules composed of long chains of monosaccharide units bound together by glycosidic linkages, and on enzymatic treatment give the constituent monosaccharides or oligosaccharides. They range in structure from linear to highly branched. Examples include storage polysaccharides such as starch and glycogen, and structural polysaccharides such as cellulose and chitin. Polysaccharides have a general formula of Cₓ(H₂O)_{y} where x is usually a large number between 200 and 2500. When the repeating units in the polymer backbone are six-carbon monosaccharides, as is often the case, the general formula simplifies to (C₆H₁₀O₅)n, where typically 40≤n≤3000. Polysaccharides contain more than ten monosaccharide units but the precise cut off varies somewhat according to convention. Polysaccharides also include callose or laminarin, chrysolaminarin, xylan, arabinoxylan, mannan, fucoidan and galactomannan.

Starch is a polymeric carbohydrate consisting of a large number of glucose units joined by glycosidic bonds. It is the most common carbohydrate in human diets and is contained in large amounts in staple foods like potatoes, wheat, maize, rice, and cassava. Pure starch is a white, tasteless and odorless powder that is insoluble in cold water or alcohol. It consists of two types of molecules: the linear and helical amylose and the branched amylopectin. Depending on the plant, starch generally contains 20 to 25% amylose and 75 to 80% amylopectin by weight.

In industry, starch is converted into sugars, for example by malting, and fermented to produce ethanol in the manufacture of beer, whisky and biofuel. It is processed to produce many of the sugars used in processed foods. Mixing most starches in warm water produces a paste, such as wheat paste, which can be used as a thickening, stiffening or gluing agent. The biggest industrial non-food use of starch is as an adhesive in the papermaking process. Starch can be applied to parts of some garments before ironing, to stiffen them.

Starch (a polymer of glucose) is used as a storage polysaccharide in plants, being found in the form of both amylose and the branched amylopectin. In animals, the structurally similar glucose polymer is the more densely branched glycogen, sometimes called “animal starch”. Glycogen's properties allow it to be metabolized more quickly, which suits the active lives of moving animals.

“Sorted”, refers to a process in which waste, such as MSW, is substantially fractionated into separate fractions such that organic material is substantially separated from plastic and/or other non-biodegradable material.

“Sorted waste” (or “sorted MSW”) as used herein refers to waste in which approximately less than 30%, preferably less than 20% and most preferably less than 15% by weight of the dry weight is not biodegradable material.

“Unsorted” refers to that the waste or the MSW is not substantially fractionated into separate fractions such that organic material is not substantially separated from plastic and/or other inorganic material, notwithstanding removal of some large objects or metal objects and notwithstanding some separation of plastic and/or other inorganic material may have taken place e.g. in front of the bioreactor. The terms “unsorted waste” (or “unsorted MSW”), as used herein, refers to waste comprising a mixture of biodegradable and non-biodegradable material in which 15% by weight or greater of the dry weight is non-biodegradable material. Waste that has been briefly sorted yet still produce a waste (or MSW) fraction that is unsorted. Typically, unsorted MSW may comprise organic waste, including one or more of food and kitchen waste; paper- and/or cardboard-containing materials; recyclable materials, including glass, bottles, cans, metals, and certain plastics; burnable materials; and inert materials, including ceramics, rocks, and debris. The recyclable material might be before or after source sorting.

“Waste” comprises, sorted and unsorted, municipal solid waste (MSW), agriculture waste, hospital waste, industrial waste, e.g., waste fractions derived from industry such as restaurant industry, food processing industry, general industry; waste fractions from paper industry; waste fractions from recycling facilities; waste fractions from food or feed industry; waste fraction from the medicinal or pharmaceutical industry; waste fractions from hospitals and clinics, waste fractions derived from agriculture or farming related sectors; waste fractions from processing of sugar or starch rich products; contaminated or in other ways spoiled agriculture products such as grain, potatoes and beets not exploitable for food or feed purposes; or garden refuse.

“Waste fractions derived from households” comprises unsorted municipal solid waste (MSW); MSW processed in some central sorting, shredding or pulping device such as e.g. Dewaster® or reCulture®; Solid waste sorted from households, including both organic fractions and paper rich fractions; RDF (Refuse-Derived-Fuel); fraction derived by post treatment as e.g. inerts, organic fractions, metals, glass, and plastic fractions. In a preferred embodiment a 2D and 3D fraction is prepared. The 2D fraction can be further separated into recyclables and/or residuals such as SRF (Solid Recovered Fuel), RDF (Refused Derived Fuel) and/or inerts. The 3D fraction can also be further separated into recyclables and/or residuals such as metals, 3D plastic and/or RDF.

“Waste fractions derived from the industry” comprises general industry waste fractions containing paper or other organic fractions now being treated as household waste; waste fraction from paper industry, e.g. from recycling facilities; waste fractions from food and feed industry; waste fractions from the medicinal industry, hospital and clinic waste, airport waste, other public and private services derived waste.

“Waste fractions derived from agriculture or farming related sectors” comprises waste fractions from processes including sugar or starch rich products such as potatoes and beet; contaminated or in other ways spoiled agriculture products such as grain, potatoes and beet not exploitable for food or feed purposes; garden refuse; manure, or manure derived products

“Waste fractions derived from municipal, county or state related or regulated activities” comprises sludge from wastewater treatment plants; fibre or sludge fractions from biogas processing; general waste fractions from the public sector containing paper or other organic fractions.

Enzyme Classes

“Enzyme” is a protein which has a catalytic function, meaning that it increases the rate of chemical reaction without undergoing any overall chemical change on itself in the process. Based on the classification by the Enzyme Commission (EC), there are six main classes of enzymes which catalyse different types of reaction, namely oxidoreductases (EC 1.X.X.X), transferases (EC 2.X.X.X), hydrolases (EC 3.X.X.X), lyases (EC 4.X.X.X), isomerases (EC 5.X.X.X) and ligases (EC 6.X.X.X). Enzymes involved in the liquefaction and/or saccharification of organic materials mostly belong to the third category (EC 3.X.X.X). These enzymes facilitate the treatment reaction, i.e. the splitting of chemical bond with the participation of water as co-substrate. The enzymes in this category are usually named according to the substrate that they hydrolyse: Amylase(s) hydrolyse starch (amylose and amylopectin), cellulase(s) hydrolyse cellulose, hemicellulase(s) hydrolyse hemicellulose, pectinase(s) hydrolyse pectins, lipase(s) hydrolyse lipids, and protease(s) hydrolyse proteins. Some of the hemicellulase(s) are esterase(s), performing catalysis on ester bonds similar as in the case of lipase(s). Some pectinase(s) are lyases which remove chemical group using non-hydrolytic reactions. Recently, a new enzyme class termed lytic polysaccharide monooxygenase (LPMO) which has catalytic activity on cellulose was discovered (Quinlan et al., 2011, Proc. Natl. Acad. Sci. USA 208: 15079-15084; Phillips et al., 2011, ACS Chem. Biol. 6: 1399-1406; Lin et al., 2012, Structure 20: 1051-1061). LPMOs catalyse oxidative cleavage of cellulose with either oxygen or hydrogen peroxide as co-substrate and were grouped under auxiliary activity 9 polypeptide. Another oxidative enzyme belonging to other class, such as catalase (EC 1.11.1.6), catalyse the conversion of hydrogen peroxide to water and oxygen.

Starch Degrading Enzymes

“Amylase” is an enzyme that catalyses the hydrolysis of starch into sugars. Important enzymes for use in hydrolysis of starch are alpha-amylases (1,4-[alpha]-1D-glucan glucanohydrolases, (EC 3.2.1.1). These are endo-acting hydrolases which cleave 1,4-[alpha]-p-glucosidic bonds and can bypass but cannot hydrolyse 1,6-alpha-D-glucosidic branchpoints. However, also exo-acting glycoamylases such as beta-amylase (EC 3.2.1.2) and pullulanase (EC 3.2.1.41) can be used for starch hydrolysis. The result of starch hydrolysis is primarily glucose, maltose, maltotriose, q-dextrin and varying amounts of oligosaccharides. Amylases include, but are not limited to, alpha-amylases derived from the genus Rhizomucor such as e.g. Rhizomucor pusillus such as e.g. the alpha-amylase encoded by SEQ ID NO: 5 as disclosed in WO17076421or homologs thereof.

Cellulose Degrading Enzymes

“Cellulase(s)” is meant to comprise one or more enzymes capable of degrading cellulose and/or related compounds. Cellulase can also be used for any mixture or complex of various such enzymes, that act serially or synergistically to decompose cellulosic material. Cellulases break down the cellulose molecule into monosaccharides (“simple sugars”) such as glucose, and/or shorter polysaccharides and oligosaccharides. Specific reactions may comprise hydrolysis of the 1,4-beta-D-glycosidic linkages in cellulose, hemicellulose, lichenin, and cereal beta-D-glucans. Several different kinds of cellulases are known, which differ structurally and mechanistically. Synonyms, derivatives, and/or specific enzymes associated with the name “cellulase” comprise endo-1,4-beta-D-glucanase (beta-1,4-glucanase, beta-1,4-endoglucan hydrolase, endoglucanase D, 1,4-(1,3,1,4)-beta-D-glucan 4-glucanohydrolase), carboxymethyl cellulase (CMCase), avicelase, celludextrinase, cellulase A, cellulosin AP, alkali cellulase, cellulase A 3, 9.5 cellulase, and pancellase SS.

Cellulases can also be classified based on the type of reaction catalysed, where endocellulases (EC 3.2.1.4) randomly cleave internal bonds at amorphous sites that create new chain ends, exocellulases or cellobiohydrolases (EC 3.2.1.91) cleave two to four units from the ends of the exposed chains produced by endocellulase, resulting in tetra-, tri-or disaccharides, such as cellobiose. Exocellulases are further classified into type I—that work processively from the reducing end of the cellulose chain, and type II—that work processively from the nonreducing end. Cellobiases (EC 3.2.1.21) or beta-glucosidases hydrolyse the exocellulase product into individual monosaccharides. Oxidative cellulases depolymerize cellulose by radical reactions, as for instance cellobiose dehydrogenase (acceptor). Cellulose phosphorylases depolymerize cellulose using phosphates instead of water. The prevalent understanding of the cellulolytic system divides the cellulases into three classes; endo-1,4-[beta]-D-glucanases (EG) (EC 3.2.1.4), which hydrolyse internal p-1,4-glucosidic bonds randomly in the cellulose chain, exo-1,4-[beta]-D-glucanases or cellobiohydrolases (CBH) (EC 3.2.1.91), which cleave off cellobiose units from the ends of cellulose chains; 1,4-[beta]-D-glucosidase (EC 3.2.1.21), which hydrolyses cellobiose to glucose and also cleaves off glucose units from cellooligosaccharides.

“Endoglucanases” means a 4-(1,3;1,4)-beta-D-glucan 4-glucanohydrolase (EC 3.2.1 0.4) that catalyzes hydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3-1,4 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). Endoglucanase activity can also be determined using carboxymethyl cellulose (CMC) as substrate according to the procedure of Ghose, 1987, Pure and Appl. Chem. 59: 257-268, at pH 5, 40° C. Endoglucanases include, but are not limited to one or more of: Acidothermus cellulolyticus endoglucanase (WO 91/05039; WO 93/15186; U.S. Pat. No. 5,275,944; WO 96/02551; U.S. Pat. No. 5,536,655; WO 00/70031; WO 05/093050), Erwinia carotovara endoglucanase (Saarilahti et al., 1990, Gene 90: 9-14), Thermobifida fusca endoglucanase III (WO 05/093050), and Thermobifida fusca endoglucanase V (WO 05/093050).

Examples of fungal endoglucanases include, but are not limited to one or more of: Trichoderma reesei endoglucanase I (Penttila et al., 1986, Gene 45: 253-263, Trichoderma reesei Cel7B endoglucanase I (GenBank:M15665), Trichoderma reesei endoglucanase II (Saloheimo et al., 1988, Gene 63:11-22), Trichoderma reesei Cel5A endoglucanase II (GenBank:M19373), Trichoderma reesei endoglucanase III (Okada et al., 1988, Appl. Environ. Microbiol. 64: 555-563, GenBank:AB003694), Trichoderma reesei endoglucanase V (Saloheimo et al., 1994, Molecular Microbiology 13: 219-228, Gen Ban k:Z33381), Aspergillus aculeatus endoglucanase (Ooi et al., 1990, Nucleic Acids Research 18: 5884), Aspergillus kawachii endoglucanase (Sakamoto et al., 1995, Current Genetics 27: 435-439), Fusarium oxysporum endoglucanase (GenBank:L29381), Humicola grisea var. thermoidea endoglucanase (GenBank:AB003107), Melanocarpus albomyces endoglucanase (GenBank:MAL515703), Neurospora crassa endoglucanase (Gen Bank:XM 324477), Humicola insolens endoglucanase V, Myceliophthora thermophila CBS 117.65 endoglucanase, Thermoascus aurantiacus endoglucanase I (Gen Bank:AF487830), Trichoderma reesei strain No. VTT-D-80133 endoglucanase (GenBank:M15665), and Penicillium pinophilum endoglucanase (WO 2012/062220).

“Cellobiohydrolases” means a 1,4-beta-D-glucan cellobiohydrolase (EC 3.2.1.91 and EC 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teeri, 1997, Trends in Biotechnology 15: 160-167; Teeri et al., 1998, Biochem. Soc. Trans. 26: 173-178). Cellobiohydrolase activity can be determined according to the procedures described by Lever et al., 1972, Anal. Biochem. 47: 273-279; van Tilbeurgh et al., 1982, FEBS Letters 149: 152-156; van Tilbeurgh and Claeyssens, 1985, FEBS Letters 187: 283-288; and Tomme et al., 1988, Eur. J. Biochem. 170: 575-581. Cellobiohydrolases include, but are not limited to one or more of: Aspergillus aculeatus cellobiohydrolase II (WO 2011/059740), Aspergillus fumigatus cellobiohydrolase I (WO 2013/028928), Aspergillus fumigatus cellobiohydrolase II (WO 2013/028928), Chaetomium thermophilum cellobiohydrolase I, Chaetomium thermophilum cellobiohydrolase II, Humicola insolens cellobiohydrolase I, Myceliophthora thermophila cellobiohydrolase II (WO 2009/042871), Penicillium occitanis cellobiohydrolase I (Gen Bank:AY690482), Talaromyces emersonii cellobiohydrolase I (GenBank:AF439936), Thielavia hyrcanie cellobiohydrolase II (WO 2010/141325), Thielavia terrestris cellobiohydrolase II (CEL6A, WO 2006/074435), Trichoderma reesei cellobiohydrolase I, Trichoderma reesei cellobiohydrolase II, and Trichophaea saccata cellobiohydrolase II (WO 2010/057086).

“Beta-glucosidases” means a beta-D-glucoside glucohydrolase (EC 3.2.1 0.21) that catalyzes the hydrolysis of terminal non-reducing beta-D-glucose residues with the release of beta-D-glucose. Beta-glucosidase activity can be determined using p-nitrophenyl-beta-D-glucopyranoside as substrate according to the procedure of Venturi et al., 2002, J. Basic Microbiol. 42: 55-66. One unit of beta-glucosidase is defined as 1 0.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20. Beta-glucosidases include, but are not limited to one or more of: beta-glucosidases from Aspergillus aculeatus (Kawaguchi et al., 1996, Gene 173: 287-288), Aspergillus fumigatus (WO 2005/047499), Aspergillus niger (Dan et al., 2000, J. Biol. Chem. 275: 4973-4980), Aspergillus oryzae (WO 02/095014), Penicillium brasilianum IBT 20888 (WO 2007/019442 and WO 2010/088387), Thielavia terrestris (WO 2011/035029), and Trichophaea saccata (WO 2007/019442).

Hemicellulose Degrading Enzymes

“Hemicellulase(s)” is meant to comprise one or more enzymes capable and/or contributing to breaking down hemicellulose, one of the major components of plant cell walls. Hemicellulose is a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. Hemicellulose can be classified based on the carbohydrate monomer that construct the backbone chain, i.e. glucan (polymer of glucose), glucomannan (polymer of glucose and mannose), mannan (polymer of mannose) and xylan (polymer of xylose). These backbone chains can have side chains of other carbohydrate monomers, acetyl group and/or glucuronic acid. Glucan backbone with no side chains and beta-1,3-1,4 linkage is termed mixed linkage beta-glucan as found in grasses. Glucan backbone with xylose side chains is termed xyloglucan which is prominent in hardwood. Glucomannan backbone with galactose substitutions as found in softwood is termed galactoglucomannan. Mannan backbone can be substituted with galactose and thus is termed galactomannan. Xylan backbone substituted mainly with glucuronic acid is termed glucuronoxylan as found in hardwood. Xylan backbone substituted with glucuronic acid, acetyl group and arabinose moiety which can be feruloylated is termed glucuronoarabinoxylan and is prominent in grasses.

The variable structure and organization of hemicelluloses require the concerted action of many enzymes for its complete degradation. The catalytic modules of hemicellulases are either glycoside hydrolases (GHs) that hydrolyze glycosidic bonds (EC 3.2.X.X), or carbohydrate esterases (CEs), which hydrolyze ester linkages of acetyl or ferulic acid side groups (EC 3.1.X.X). Hemicellulases are collectively named after the backbone chains that they hydrolyze and specifically according to the bonds and side chains that they cleave or remove, respectively. Beta-glucanase(s) hydrolyse mixed linkage (beta-1,3-1,4) beta-glucans, whereas xyloglucanase(s) hydrolyse xyloglucans. Glucomannanase(s) and mannanase(s) hydrolyse (galacto-) glucomannans and (galacto-) mannans, respectively. In a similar manner, glucuronoxylanase(s) and xylanase(s) are collective terms for enzymes that hydrolyse glucuronoxylan and xylan, respectively. The enzymes that hydrolyse glucuronoarabinoxylan can be termed arabinoxylanase as in the case of glucuronoxylanase(s), though it consists of xylanase(s) and other enzymes which remove side chain groups. The latter group consists of alpha-arabinofuranosidase which removes arabinose side chain, alpha-glucuronidase which removes glucuronic acid side chain as well as esterase(s) such as acetyl xylan esterase and feruloyl esterase which remove acetyl and feruroyl groups, respectively.

“Beta-glucanase(s)” means any type of endo-beta-glucanase that hydrolyzes (1,3)- or (1,4)-linkages in beta-D-glucans (EC 3.2.1 0.73) (EC 3.2.1 0.6). Beta-glucanases includes but are not limited to beta-glucanases derived from a member of the genus Aspergillus such as e.g. Aspergillus aculeatus such as e.g. the beta-glucanase encoded by the sequence encoded by SEQ ID NO: 4 as disclosed in WO17076421 or homologs thereof.

“Xyloglucanase(s)” is meant to comprise one or more enzymes capable of degrading xyloglucan and/or related compounds, comprising e.g. xyloglucan-specific endo-beta-1,4-glucanase (EC 3.2.1.151). This enzyme belongs to the family of hydrolases, specifically those glycosidases that hydrolyse O- and S-glycosyl compounds. Other names in common use may include XEG, xyloglucan endo-beta-1,4-glucanase, xyloglucanase, xyloglucanendohydrolase, XH, and 1,4-beta-D-glucan glucanohydrolase.

“Mannanase(s)” means a beta-mannanase and defined as an enzyme belonging to EC 3.2.1.78 or EC 3.2.1.25. Mannanase also comprises endo-mannanase and/or 1,4-beta-mannanase. Mannanases have been identified in several Bacillus organisms. For example, Talbot et al., Appl. Environ. Microbiol., Vol.56, No. 11, pp. 3505-3510 (1990) describes a beta-mannanase derived from Bacillus stearothermophilus having an optimum pH of 5.5-7.5. Mendoza et al., World J. Microbiol. Biotech., Vol. 10, No. 5, pp. 551-555 (1994) describes a beta-mannanase derived from Bacillus subtilis having an optimum activity at pH 5.0 and 55° C. JP-03047076 discloses a beta-mannanase derived from Bacillus sp., having an optimum pH of 8-10. JP-63056289 describes the production of an alkaline, thermostable beta-mannanase. JP-08051975 discloses alkaline beta-mannanases from alkalophilic Bacillus sp. AM-001. A purified mannanase from Bacillus amyloliquefaciens is disclosed in W097/1 1164. WO 94/25576 discloses an enzyme from Aspergillus aculeatus, CBS 101 0.43, exhibiting mannanase activity and WO 93/24622 discloses a mannanase isolated from Trichoderma reesei.

“Glucomannanase(s)” is meant to comprise one or more enzymes capable of degrading glucomannans and/or related compounds. This includes endo-1,4-[beta]-D-mannanases (EC 3.2.1.78) which cleave the bond between mannosyl moieties in the backbone, beta-glucosidases (EC 3.2.1 0.21) which cleave the bond between glucosyl and mannosyl moieties in the backbone and [alpha]-D-galactosidases (EC 3.2.1.22) which removes the galactose side chains from the backbone.

“Mannosidase(s)” means a 1,4-[beta]-D-mannosidases (EC 3.2.1.25), which cleave mannooligosaccharides to mannose. The enzyme can be derived from the genus Bacillus such as e.g. Bacillus bogoriensis such as e.g. the endo-mannosidase encoded by SEQ ID NO: 6 as disclosed in WO17076421 or homologs thereof.

“Xylanase(s)” means a 1,4-beta-D-xylan-xylohydrolase (EC 3.2.1.8) that catalyzes the endohydrolysis of 1,4-beta-D-xylosidic linkages in xylans. One unit of xylanase activity is defined as 1.0 μmole of azurine produced per minute at 37° C., pH 6 from 0.2% AZCL-arabinoxylan as substrate in 200 mM sodium phosphate pH 6. Xylanases comprise one or more enzymes capable of degrading xylan and/or related compounds. Xylanase is any of several enzymes produced e.g. by microorganisms such as yeast that catalyse decomposition of xylan and/or related polysaccharides. Xylanase can also be used for any mixture or complex of various such enzymes that act serially or synergistically to decompose xylanosic material. Synonyms, derivatives, and specific enzymes associated with the name “xylanase” may comprise EC 3.2.1.8, endo-(1->4)-beta-xylan 4-xylanohydrolase, endo-1,4-xylanase, endo-1,4-beta-xylanase, beta-1,4-xylanase, endo-1,4-beta-D-xylanase, 1,4-beta-xylan xylanohydrolase, beta-xylanase, beta-1,4-xylan xylanohydrolase, beta-D-xylanase and/or xylosidase capable of degrading xylan, such as beta-1,4-xylan into xylose, thus contributing to breaking down hemicellulose, one of the major components of plant cell walls.

“Glucuronoxylanase(s)” is meant to comprise one or more enzymes capable of degrading glucuronoxylan and/or related compounds.

“Xylosidases” means the enzyme xylan 1,4-beta-xylosidase (EC 3.2.1.37) which is also named xylobiase, beta-xylosidase, exo-1,4-beta-D-xylosidase or 4-beta-D-xylan xylohydrolase. This enzyme catalyses the hydrolysis of (1,4)-beta-D-xylans removing successive D-xylose residues from the non-reducing termini of the substrate, e.g. hemicellulose and the disaccharide xylobiose. One unit of beta-xylosidase is defined as 1.0 μmole of p-nitrophenolate anion produced per minute at 40° C., pH 5 from 1 mM p-nitrophenyl-beta-D-xyloside in 100 mM sodium citrate containing 0.01% TWEEN® 20.

“Alpha-L-arabinofuranosidase” means an alpha-L-arabinofuranoside arabinofurano-hydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinosidase, alpha-arabinosidase, alpha-L-arabinosidase, alpha-arabinofuranosidase, polysaccharide alpha-L-arabinofuranosidase, alpha-L-arabinofuranoside hydrolase, L-arabinosidase, or alpha-L-arabinanase.

“Alpha-glucuronidase” means an alpha-D-glucosiduronate glucuronohydrolase (EC 3.2.1 0.139) that catalyzes the hydrolysis of an alpha-D-glucuronoside to D-glucuronate and an alcohol. Alpha-glucuronidase activity can be determined according to de Vries, 1998, J. Bacteriol. 180: 243-249. One unit of alpha-glucuronidase equals the amount of enzyme capable of releasing 1 μmole of glucuronic or 4-O-methylglucuronic acid per minute at pH 5,

“Esterase(s)” is meant to comprise one or more enzymes that catalyse the hydrolysis of organic esters to release an alcohol or thiol and acid. The term could be applied to enzymes that hydrolyse carboxylate, phosphate and sulphate esters, but is more often restricted to the first class. Examples of esterases comprise acetylesterases and feruloyl esterase, e.g. EC 3.1.X.X.

“Acetylxylan esterase” means a carboxylesterase (EC 3.1.1.72) that catalyzes the hydrolysis of acetyl groups from polymeric xylan, acetylated xylose, acetylated glucose, alpha-naphthyl acetate, and p-nitrophenyl acetate. One unit of acetylxylan esterase is defined as the amount of enzyme capable of releasing 1 μmole of p-nitrophenolate anion per minute at pH 5, 25° C.

“Feruloyl esterase(s)” means a 4-hydroxy-3-methoxycinnamoyl-sugar hydrolase (EC 3.1.1.73) that catalyzes the hydrolysis of 4-hydroxy-3-methoxycinnamoyl (feruloyl) groups from esterified sugar, which is usually arabinose in natural biomass substrates, to produce ferulate (4-hydroxy-3-methoxycinnamate). Feruloyl esterase (FAE) is also known as ferulic acid esterase, hydroxycinnamoyl esterase, FAE-III, cinnamoyl ester hydrolase, FAEA, cinnAE, FAE-I, or FAE-II. One unit of feruloyl esterase equals the amount of enzyme capable of releasing 1 μmole of p-nitrophenolate anion per minute at pH 5, 25° C.

Pectin Degrading Enzymes

“Pectinase(s)” means any enzyme that catalyzes the degradation of pectin, a polysaccharide found in plant cell walls, including 1) pectin lyase, other names pectin trans-eliminase; endo-pectin lyase; polymethylgalacturonic transeliminase; pectin methyltranseliminase; pectolyase; PL; PNL; PMGL (EC 4.2.2.10) making eliminative cleavage of (1—>4)-alpha-D-galacturonan methyl ester to give oligosaccharides with 4-deoxy-6-O-methyl-alpha-D-galact-4-enuronosyl groups at their non-reducing ends, 2) pectin pectylhydrolase, other names pectin demethoxylase; pectin methoxylase; pectin methylesterase; pectase; pectin methyl esterase; pectinoesterase (EC 3.1.1.11) hydrolyzing the methyl ester bond in pectin and 3) polygalacturonase (EC 3.2.1.15) hydrolyze the α-1,4-glycosidic linkages in polygalacturonic acid chains.

Lipid Degrading Enzymes

“Lipase” means any enzyme that catalyzes the degradation of lipids and/or having hydrolytic activity in class EC 3.1.1.-as defined by Enzyme Nomenclature. Particular useful is triacyl glycerol lipases (EC 3.1.1.3) and phospholipase A1 (EC 3.1.1.32) and phospholipase A2 (EC 3.1.1.4), but also other phospholipases (EC 3.1.1.5), (EC 3.1.4.4), (EC 3.1.4.11), (EC 3.1.4.50), (EC 3.1.4.54). Lipases include, but are not limited to, lipases derived from the genus Thermomyces sp. such as e.g. Thermomyces lanuginosus such as e.g. the lipase encoded by SEQ ID NO: 2 as disclosed in WO17076421 (or homologues thereof) or wherein the lipase is derived from the genus Humicola sp. such as e.g. Humicola insolens (or homologues thereof).

Protein Degrading Enzymes

“Protease” means any protease or proteolytic enzyme suitable for use under neutral or acidic conditions. Suitable proteases include those of animal, vegetable or microbial origin.

Chemically or genetically modified mutants are included. Suitable proteases includes metallo endoprotease that hydrolyzes internal peptide bonds (EC 3.4.24.28), serine endoprotease that hydrolyzes internal peptide bonds (EC 3.4.23.23), endoprotease that hydrolyzes peptide bonds at the carboxy side of lysine and arginine residues EC 3.4.21.4), aminopeptidase (EC 3.4.11.1) and exopeptidase that liberates amino acids by hydrolysis of the N-terminal peptide bond (EC 3.4.11.1). Proteases may be derived from the genus Bacillus, such as e.g. Bacillus amyloliquefaciens such as e.g. the protease encoded by SEQ ID NO:1 as disclosed in WO17076421, or homologues thereof.

Oxidative Enzymes

“Auxiliary Activity 9 polypeptide” or “AA9 polypeptide” means a polypeptide classified as a lytic polysaccharide monooxygenase (Quinlan et al., 2011, Proc. Natl. Acad. Sci. USA 208: 15079-15084; Phillips et al., 2011, ACS Chem. Biol. 6: 1399-1406; Lin et al., 2012, Structure 20: 1051-1061). AA9 polypeptides were formerly classified into the glycoside hydrolase Family 61 (GH61) according to Henrissat, 1991, Biochem. J. 280: 309-316, and Henrissat and Bairoch, 1996, Biochem. J. 316: 695-696. AA9 polypeptides enhance the hydrolysis of a cellulosic material by an enzyme having cellulolytic activity. Cellulolytic enhancing activity can be determined by measuring the increase in reducing sugars or the increase of the total of cellobiose and glucose from the hydrolysis of a cellulosic material by cellulolytic enzyme.

“Catalase” means a hydrogen-peroxide:hydrogen-peroxide oxidoreductase (EC 1.11.1.6). For purposes of the present invention, catalase activity is determined according to U.S. Patent No. One unit of catalase activity equals the amount of enzyme that catalyzes the oxidation of 1 μmole of hydrogen peroxide under the assay conditions.

Enzyme-Related Terms

“Cellulase activity” refers to enzymatic hydrolysis of 1,4-[beta]-D-glycosidic linkages in cellulose. In isolated cellulase enzyme preparations obtained from bacterial, fungal or other sources, cellulase activity typically comprises a mixture of different enzyme activities, including endoglucanases and exoglucanases (also termed cellobiohydrolases), which respectively catalyse endo- and exo-hydrolysis of 1,4-[beta]-D-glycosidic linkages, along with [beta]-glucosidases, which hydrolyse the oligosaccharide products of exoglucanase hydrolysis to monosaccharides. Complete treatment of insoluble cellulose typically requires a synergistic action between the different activities.

“Cellulolytic background composition (CBC) or Cellulolytic Enzyme Blend” means an enzyme composition comprising a mixture of two or more cellulolytic enzymes. The CBC may comprise two or more cellulolytic enzymes selected from: i) an Aspergillus fumigatus cellobiohydrolase I; (ii) an Aspergillus fumigatus cellobiohydrolase II; (iii) an Aspergillus fumigatus beta-glucosidase or variant thereof; and (iv) a Penicillium sp. GH61 polypeptide having cellulolytic enhancing activity; or homologs thereof. The CBC may further comprise one or more enzymes selected from: (a) an Aspergillus fumigatus xylanase or homolog thereof, (b) an Aspergillus fumigatus beta-xylosidase or homolog thereof; or (c) a combination of (a) and (b) (as described in further detail in WO 2013/028928). The major activities of the CBC may comprise: endo-1,4-beta-glucanases (E.C. 3.2.1.4); endo-1,4-beta-xylanases (E.C. 3.2.1.8); endo-1,4-beta-mannanase (E.C. 3.2.1.78), beta-mannosidase (E.0 3.2.1.25), whereas other enzymatic activities may also be present in the CBC such as activity from glucanases, glucosidases, cellobiohydrolase I cellobiohydrolase II; beta-glucosidase; beta-xylosidase; beta-L-arabinofuranosidase; amyloglucosidase; alpha-amylase; acetyl xylan esterase. The CBC may be any CBC described in WO 2013/028928 (the content of which is hereby incorporated by reference). The CBC may be from T. reesei. The CBC may be from Myceliophtora thermophilae. The CBC may be Cellic® CTec3 obtainable from Novozymes A/S (Bagsvaerd, Denmark). Cellulolytic enzyme activity can be determined by measuring the increase in production/release of sugars during hydrolysis of a cellulosic material by cellulolytic enzyme(s) under the following conditions: 1-50 mg of cellulolytic enzyme protein/g of cellulose in pre-treated corn stover (PCS) (or other pre-treated cellulosic material) for 3-7 days at a suitable temperature such as 40° C.-80° C., e.g., 40° C., 45° C., 50° C., 55° C., 60° C., 65° C., 70° C., or 80° C., and a suitable pH, such as 4-9, e.g., 4.0, 4.5, 5.0, 5.5, 6.0, 6.5, 7.0, 7.5, 8.0, 8.5, or 9.0, compared to a control treatment without addition of cellulolytic enzyme protein.

“Commercially available cellulase preparation optimized for biomass conversion” refers to a commercially available mixture of enzyme activities which is sufficient to provide enzymatic treatment of biomass such as lignocellulosic biomass and which usually comprises endocellulase (endoglucanase), exocellulase (exoglucanase), endoxylanase, acetyl xylan esterase, xylosidase and/or beta-glucosidase activities. The term “optimized for biomass conversion” refers to a product development process in which enzyme mixtures have been selected and/or modified for the specific purpose of improving yields and/or reducing enzyme consumption in treatment of biomass to fermentable sugars. A commercially available cellulase preparation optimized for biomass conversion can be used, such as one that is e.g. provided by Genencor (now DuPont), DSM or Novozymes. Usually, such compositions comprise cellulase(s) and/or hemicellulase(s), such as one or more of exoglucanases, endoglucanases, endoxylanases, xylosidases, acetyl xylan esterases and beta-glucosidases, including any combination thereof. Such enzymes can e.g. be isolated from fermentations of genetically modified Trichoderma reesei, such as, for example, the commercial cellulase preparation sold under the trademark ACCELLERASE TRIO™ from DuPont (and/or Genencor). A commercially available cellulase preparation optimized for biomass conversion that can be used is provided by Novozymes and comprises exoglucanases, endoglucanases, endoxylanases, xylosidases, acetyl xylan esterases and beta-glucosidases, such as, for example, the commercial cellulase preparations sold under either of the trademarks Cellic® CTec2 or Cellic® CTec3 from Novozymes.

It is believed that the specific enzyme activities present in different commercially available cellulase preparations optimized for biomass conversion can be analysed in detail using methods known in the art, enabling accurate measurement of degradation of the substrate that is directly correlated to the enzyme activity/concentration, such as Glycospot™

Suitable cellulase preparations optimized for biomass conversion usually comprise multiple enzyme activities, including exoglucanase, endoglucanase, hemicellulases (including xylanases) and 6-glucosidases. Enzyme preparations can be expressed in different activities/units, such as carboxymethycellulase (CMC U) units, acid birchwood xylanase units (ABXU), and pNP-glucosidase units (pNPG U). For example, ACCELLERASE TRIO™ comprises: endoglucanase activity: 2000-2600 CMC U/g, xylanase activity: >3000 ABX U/g, and beta-glucosidase activity:>2000 pNPG U/g; wherein one CMC unit of activity liberates 1 μmol of reducing sugars (expressed as glucose equivalents) in one minute at 50° C. and pH 4.8; one ABX unit is defined as the amount of enzyme required to generate 1 μmol of xylose reducing sugar equivalents per minute at 50° C.; and pH 5.3; and one pNPG unit denotes 1 μmole of nitro-phenol liberated from para-nitrophenyl-[beta]-D-glucopyranoside per minute at and pH 4.8. In order to find out how much enzyme of a given enzymatic composition should be added, a solubilization test (described below) of the enzyme composition on model waste may be applied to provide an optimum enzymatic liquefaction process.

“Microbial enzymes”, includes any enzyme such as cellulase(s), hemicellulase(s) and/or starch degrading enzyme(s), that can be expressed in suitable microbial hosts using methods known in the art. Such enzymes are also commercially available, either in pure form or in enzyme cocktails. Specific enzyme activities can be purified from commercially available enzyme cocktails, again using methods known in the art—see e.g. Sorensen et al. (2005) “Efficiencies of designed enzyme combinations in releasing arabinose and xylose from wheat arabinoxylan in an industrial fermentation residue” (Enzyme and Microbial Technology 36 (2005) 773-784), where a Trichoderma reesei beta-xylosidase is purified from Celluclast (Finizym), and further commercial enzyme preparations are disclosed.

“Xylan degrading activity” or “xylanolytic activity” means a biological activity that hydrolyzes xylan-containing material. The two basic approaches for measuring xylanolytic activity include: (1) measuring the total xylanolytic activity, and (2) measuring the individual xylanolytic activities (e.g., endoxylanases, beta-xylosidases, arabinofuranosidases, alpha-glucuronidases, acetylxylan esterases, feruloyl esterases, and alpha-glucuronyl esterases). Recent progress in assays of xylanolytic enzymes was summarized in several publications including Biely and Puchard, 2006, Journal of the Science of Food and Agriculture 86(11): 1636-1647; Spanikova and Biely, 2006, FEBS Letters 580(19): 4597-4601; Herrimann et al., 1997, Biochemical Journal 321: 375-381. Total xylan degrading activity can be measured by determining the reducing sugars formed from various types of xylan, including, for example, oat spelt, beechwood, and larchwood xylans, or by photometric determination of dyed xylan fragments released from various covalently dyed xylans. A common total xylanolytic activity assay is based on production of reducing sugars from polymeric 4-O-methyl glucuronoxylan as described in Bailey et al, 1992, Interlaboratory testing of methods for assay of xylanase activity, Journal of Biotechnology 23(3): 257-270. Xylanase activity can also be determined with 0.2% AZCL-arabinoxylan as substrate in 0.01% TRITON® X-100 and 200 mM sodium phosphate pH 6 at 37° C. One unit of xylanase activity is defined as 1 0.0 μmole of azurine produced per minute at 37° C., pH 6 from 0.2% AZCL-arabinoxylan as substrate in 200 mM sodium phosphate pH 6. Xylan degrading activity can be determined by measuring the increase in hydrolysis of birchwood xylan (Sigma Chemical Co., Inc., St. Louis, MO, USA) xylan-degrading enzyme(s) under the following typical conditions: 1 ml reactions, 5 mg/ml substrate (total solids), 5 mg of xylanolytic protein/g of substrate, 50 mM sodium acetate pH 5, 24 hours, sugar analysis using p-hydroxybenzoic acid hydrazide (PHBAH) assay as described by Lever, 1972, Anal. Biochem. 47: 273-279.

“Lactic acid producing bacteria” comprises lactic acid bacteria (LAB) where the currently accepted taxonomy is based on the List of Prokaryotic names with Standing in Nomenclature (LPSN)—an online database that maintains information on the naming and taxonomy of prokaryotes, following the taxonomy requirements and rulings of the International Code of Nomenclature of Bacteria. The phylogeny of the order is based on 16S rRNA-based LTP release 106 by ‘The All-Species Living Tree’ Project. In addition to bacteria belonging to the LAB order, the term “lactic acid producing bacteria” used herein also comprises bacteria that do not belong to the LAB order, but that are nevertheless capable of producing lactic acid. The amount of lactic acid bacteria can be measured with Assay II.

Process Related Terms

“Bioliquid” is the liquefied and/or saccharified degradable components obtained by enzymatic treatment of waste comprising organic matter. Bioliquid also refers to the liquid fraction obtained by enzymatic treatment of waste comprising organic matter once separated from non-fermentable solids. Bioliquid comprises water and organic substrates such as protein, fat, galactose, mannose, glucose, xylose, arabinose, lactate, acetate, ethanol and/or other components, depending on the composition of the waste (the components such as protein and fat can be in a soluble and/or insoluble form). Bioliquid comprises also fibres, ashes and inert impurities. The resulting bioliquid comprising a high percentage of microbial metabolites provides a substrate for gas production, a substrate suitable for anaerobic digestion e.g. for the production of biogas.

“Biogas” is the mixture of gases produced by the breakdown of organic matter in the absence of oxygen. Biogas may be produced from raw materials such as agricultural waste, manure, municipal waste, plant material, sewage, green waste or food waste. Biogas is a renewable energy source.

Biogas is produced by anaerobic digestion with methanogen or anaerobic organisms, which digest material inside a closed system, or fermentation of biodegradable materials. This closed system is called an anaerobic digester, biodigester or a bioreactor.

Biogas is primarily methane (CH₄) and carbon dioxide (CO₂) and may have small amounts of hydrogen sulfide (H₂S), moisture and siloxanes. The gases methane, hydrogen, and carbon monoxide (CO) can be combusted or oxidized with oxygen. This energy release allows biogas to be used as a fuel; it can be used for any heating purpose, such as cooking. It can also be used in a gas engine to convert the energy in the gas into electricity and heat.

By the term “bioreactor” is meant equipment or a system supporting a biologically active environment, e.g. an environment, where biological processes are carried out, i.e. processes involving microorganism or biochemically active substances derived from microorganisms. One example of a bioreactor is a container or a vessel in which the microorganisms and/or biochemically active substances kept at desired conditions, which allow the bioreactions to run, e.g. aerobic or anaerobic conditions, temperature etc.

“Dry matter,” also appearing as “DM”, refers to total solids, both soluble and insoluble, and effectively means “non-water content.” Dry matter content is measured by drying at approximately 60° C. for 48 hours as described in Assay VIII.

“Hydrolysis” is the splitting of chemical bond with the participation of water as co-substrate. The term is applied when municipal solid waste material is treated with an enzyme composition to break down cellulose and/or hemicellulose and other substrates to fermentable sugars, such as glucose, cellobiose, xylose, xylulose, arabinose, mannose, galactose, and/or soluble oligosaccharides (also known as saccharification). The enzymatic treatment is performed by one or more enzyme compositions in one or more stages. In the present disclosure, the terms “hydrolyzation”, “liquefaction”, “saccharification” and “solubilization” may be used interchangeably.

The enzymatic treatment can be carried out as a batch process or series of batch processes. The enzymatic treatment can be carried out as a fed batch or continuous process, or series of fed batch or continuous processes, where the municipal solid waste material is fed gradually to, for example, a solution containing an enzyme composition. The enzymatic treatment may be continuous in which an MSW material and an enzyme composition are added at different intervals throughout the treatment and the hydrolysate is removed at different intervals throughout the enzymatic treatment. The removal of the hydrolysate may occur prior to, simultaneously with, or after the addition of the cellulosic material and the cellulolytic enzymes composition.

An “effective amount” of one or more isolated enzyme preparations is an amount where collectively the enzyme preparation used achieves sufficient solubilization of waste to provide a solution comprising a high percentage of sugars and other soluble degradation products, a substrate suitable for anaerobic digestion e.g. for the production of biogas. The effective amount can be determined by use of a solubilization test as described herein.

Enzymatic treatment is preferably carried out in a suitable aqueous environment under conditions that can be readily determined by one skilled in the art. In one aspect, enzymatic treatment is performed under conditions suitable for the activity of the enzymes(s), i.e., optimal for the enzyme(s).

“Solubilization test” is a test applied in order to find out how much of a given enzymatic composition should be added to the waste for sufficient enzymatic treatment. A solubilization test of the selected enzyme composition on MSW model substrate can be applied to identify an optimum enzymatic solubilization process. The solubilization of the waste, such as municipal solid waste, can be determined by applying the below testing method:

A model substrate consisting of 41% mixed food waste of vegetable origin, 13% mixed food waste of animal origin and 46% mixed cellulosic waste is shredded, mixed and milled several times until homogeneous, passed through a 3 mm screen, divided into smaller portions and stored frozen at −18° C.

A set of pre-tared 50 mL centrifuge tubes, each containing 1.500±0.010 g TS (Total Solids at 60° C.) of the above mentioned model substrate in a 50 mM Sodium acetate buffer pH 4.50±0.05, are added various amounts of the enzyme to test (typically 5-60 mg EP/g TS of model substrate) for a final total weight of 20.000±0.025 g in each tube.

The tubes are closed with tight fitting lids and the reaction mixtures are incubated at 50±1° C. for 24 hours ±10 minutes with agitation by inverting the test tubes (end-over-end) at 10.0±0.5 revolutions per minute.

Immediately after finished incubation the tubes are centrifuged at 2100±10 G for 10 minutes, and immediately after centrifugation (and within less than 5 minutes) the supernatant is decanted into another set of pre-tared tubes. The first set of tubes (including lids), with the residual undissolved model substrate, and the second set of tubes, with the decanted supernatant containing the solubilized model substrate, are weighed on a 4 decimal analytical balance and then left to dry at 60±1° C. for 6 days in a well-ventilated drying cabinet. After drying the tubes (including lids) are weighed again, the TS amounts in pellet and supernatant are determined and the mass balance is calculated as: Mass balance %=((TS pellet+TS supernatant−TS Enzyme)/TS model substrate)*100%

The mass balance based on TS model substrate (1.500±0.010 g), to assure for no loss of material and proper drying, will typically be in the interval of 95-105%.

Based on the Total amount and TS amount of the decanted supernatant, TS % in the decanted supernatant is calculated as:

TS %=(TS decanted supernatant/Total decanted supernatant)*100%

Finally, the solubilization is calculated as:

Solubilization %=(((TS %*Actual water/(1−TS %))−TS Enzyme)/TS model substrate)* 100%

By calculating solubilization based on TS % of the decanted supernatant and the Actual water amount (actual weight of decanted supernatant and wet pellet, subtracted initial weight of TS in model substrate added), the liquid phase that is trapped in the centrifugation pellet will also be accounted for.

A graph of solubilization versus enzyme dose will show the characteristics of enzyme efficacy (maximum solubilization at high enzyme dosages) and enzyme potency (dose required for obtaining a certain level of solubilization).
Enzyme efficacy may typically be 35-70% solubilization, depending on the model substrate composition and the enzyme composition to test. Dose in use may typically be defined to obtain 85-95% of the efficacy.

“Isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance).

“Pre-treatment” means any pre-treatment process known in the art can be used to disrupt plant cell wall components of the municipal solid waste material (Chandra et al., 2007, Adv. Biochem. Engin./Biotechnol. 108: 67-93; Galbe and Zacchi, 2007, Adv. Biochem. Engin./Biotechnol. 108: 41-65; Hendriks and Zeeman, 2009, Bioresource Technology 100: Mosier et al., 2005, Bioresource Technology 96: 673-686; Taherzadeh and Karimi, 2008, Int. J. Mol. Sci. 9: 1621-1651; Yang and Wyman, 2008, Biofuels Bioproducts and Biorefining-Biofpr. 2: 26-40). Conventional pre-treatments include, but are not limited to, steam pre-treatment (with or without explosion), dilute acid pre-treatment, hot water pre-treatment, alkaline pre-treatment, lime pre-treatment, wet oxidation, wet explosion, ammonia fiber explosion, organosolv pre-treatment, and biological pre-treatment. Additional pre-treatments include ammonia percolation, ultrasound, electroporation, microwave, supercritical CO₂, supercritical H₂O, ozone, ionic liquid, bricketing, pelleting and gamma irradiation pre-treatments.

“Solubilization” means enzymatic treatment of a waste resulting in liquefaction and/or saccharification of organic matter. In present disclosure, the terms “hydrolyzation”, “liquefaction”, “saccharification” and “solubilization” may be used interchangeably.

“Sanitization” is the process of reducing the number of microorganisms to a level that has been officially approved as safe. It is the control bacterial levels in equipment and utensils found in dairies, other food-processing plants, eating and drinking establishments, and other places in which no specific pathogenic microorganisms are targeted. In the present document, a strain of E. coli is used as hygiene indicator and a result of <10² CFU of E. coli per gram of waste is considered as being satisfactory, i.e. as sanitized waste (Source: “Guidelines for assessing the microbiological safety of ready-to-eat foods placed on the market”, Health Protection Agency, Nov 2009, p.24).

“2D/3D Separation” is achieved in one or more steps. In one embodiment first, a ballistic separator removes two streams of non-degradable materials, producing a 2D fraction comprising plastic bags and other generally formless material, a 3D fraction comprising bottles and containers having a definite shape, and a volume of a biogenic liquid slurry of bio-degradable components. In a second step of this embodiment, the 2D fraction is further subject to pressing with a screw press or similar device to further increase the yield of the biogenic slurry. The 2D fraction may be further subject to washing, in order to further recover bio-degradable material.

### BRIEF DESCRIPTION OF DRAWINGS

FIG. 1 is a schematic overview of an example of a waste process comprising a bioreactor wherein the self-sanitizing enzymatic and microbial treatment takes place

FIG. 2 shows bacterial counts on metal from Mechanical Biological Treatment (MBT) (black bars) and from the process of the invention (grey bars).

FIG. 3 shows bacterial count for Refused Derived Fuel (RDF) from Mechanical Biological Treatment (MBT) (black bars) and from the process of the invention (grey bars).

FIG. 4 shows a contour plot showing the negative logarithm with base 10 of relative reduction of CFU counts after 24 h (relative to 0 h). The black points show the experimentally tested conditions and a digit next to the points shows the number of replicates performed for a specific combination of pH and temperature. If no digit is shown next to a point, then this combination of pH and temperature was tested only once. Each contour line shows the 10ⁿ (where n is a number in a box crossed by the contour) times reduction of relative CFU counts after 24 h according to a model: (−log₁₀(relative reduction of CFU counts))^{0.59}=9.2615−0.9346×pH−+0.00274×T²

### DETAILED DESCRIPTION OF THE INVENTION

The present invention pertains to a method for sanitizing waste, the method comprising:
a) Subjecting waste comprising biodegradable material and non-biodegradable material and having a total bacterial count of at least 2.5×10⁸ CFU/gram waste, a bacterial count of E. coli of at least 1.5×10⁶ CFU/gram waste or a bacterial count of Enterobacteriaceae of at least 1.5×10⁸ CFU/gram waste, to enzymatic and/or microbial treatment in a bioreactor at a pH between 3.0 and 6.0 and at a temperature of between 40° C. and 60° C. for a period of 10 to 30 hours to obtain at least partial reduction in bacterial count.

The method may further comprise the pre-step:
a) Removal of large items, shredding and/or pulping.

The method may further comprise the subsequent steps:
b) subjecting the treated waste from step a) to one or more separation step(s), whereby a bioliquid and a solid fraction is provided;
c) subjecting said bioliquid and/or solid fraction to downstream processing

Downstream processing could be any process involving the solid or the liquid fraction of the waste obtained from step b) which takes place downstream of the enzymatic and/or microbial treatment in the bioreactor in step a). Examples of downstream processes are washing processes, evaporation processes, collection of bioliquid or part of the bioliquid obtained in step b) and anaerobic digestion. Downstream process also includes processes wherein the solid and/or liquid fraction of the waste obtained from step b) is converted into biogas, which can be combusted to generate electricity and/or heat, and processes wherein the solid and/or liquid fraction of the waste obtained from step b) is converted into renewable natural, biomethane gas and/or transportation fuels.

The inventors have surprisingly found that when reacting a waste fraction with a specific content of natural occurring bacteria and enzyme at low temperatures (40° C.-60° C.), the resulting bioliquid and non-biodegradable waste material has very low numbers of pathogenic bacteria. As a result, the bioliquid, the waste and the equipment used in waste treatment do not expose the environment, e.g. the workers, to undesired bacteria.

Low temperatures during reaction with enzymes are advantageous as fuel for heating the waste fraction to high temperatures, e.g. 75° C., is saved. Considerable savings are available when waste fractions are reacted with enzymes for about 10 to 30 hours. A further advantage is that handling a process at low temperature is easier than handling a process performed at high temperatures.

The inventors have found that even when reacting the waste at low temperatures with enzymes, the resulting bioliquid and non-biodegradable waste material has very low numbers of bacteria, e.g. pathogenic bacteria. The number of bacteria present on the waste may be reduced to a bacterial count of E. coli of less than 20 CFU/gram waste and/or a bacterial count of Enterobacteriaceae of less than 10² CFU/gram waste.

The inventive method for producing a bioliquid comprises under step a) subjecting waste comprising biodegradable and non-biodegradable material to enzymatic and/or microbial treatment. The waste comprises biodegradable material, which is organic material that can be hydrolysed by enzymes and/or microorganisms. The organic material may comprise carbohydrates, proteins, fat and mixtures thereof, which are organic matter that are typical present in household waste. The waste further comprises material that is not biodegradable, such as plastic or metal.

The waste can be unsorted. In an embodiment of the invention the unsorted waste comprises a mixture of biodegradable and non-biodegradable material in which 15% by weight or greater of the dry weight is non-biodegradable material.

In an embodiment of the invention, the waste comprises a mixture of biodegradable and non-biodegradable material in which at least 20% w/w is non-biodegradable material, based on the weight of the waste. In one embodiment, at least 25% of the waste is non-biodegradable material, at least 30% of the waste is non-biodegradable material, at least 35% of the waste is non-biodegradable material, at least 40% of the waste is non-biodegradable material, at least 45% of the waste is non-biodegradable material or at least 50% of the waste is non-biodegradable material.

The waste can be municipal solid waste (MSW), e.g. city waste or waste disposed from domestic household and public facilities. The waste comprises a natural microflora, which has a total bacterial count of at least 2.5×10⁸ CFU/gram waste, a bacterial count of E. coli of at least 1.5×10⁶ CFU/gram waste and/or a bacterial count of Enterobacteriaceae of at least 1.5×10⁸ CFU/gram waste. The natural microflora may comprise lactic acid bacteria, which may proliferate during the time period, where the waste is subjected to the enzyme composition. In a preferred embodiment of the invention the waste comprises a natural microflora, which has a total bacterial count of at least 3.0×10⁸ CFU/gram waste, a bacterial count of E. coli of at least 1.6×10⁶ CFU/gram waste and/or a bacterial count of Enterobacteriaceae of at least 1.9×10⁸ CFU/gram waste.

It was previously believed that, in order to produce bioliquid from waste, inoculation of bacteria to the waste fraction was necessary. The inventors have found that the bacteria naturally occurring in the waste fraction are enough to control the microflora during the reaction time, where the waste fraction is exposed to the enzyme composition. The Examples show that the numbers of Enterobacteriaceae and E. coli in the bioliquid are very low,

In one embodiment of the invention, the waste provided contain lactic acid bacteria. The waste may have a ratio between the lactic acid bacteria and the total bacterial count of at least 1:1, such as at least 1:1.5, at least 1:2, at least 1:3, at least 1:4, at least 1:5 or at least 1:10.

The waste fraction provided in the inventive method may have a dry matter content in the range of 10-90% w/w. The content of dry matter in the waste fraction can be measured by Assay VIII. In one embodiment of the invention, the waste fraction may have a dry matter content in the range of 30-80% w/w, preferably in the range of 50-70% w/w.

In one embodiment of the invention the waste fraction provided in the inventive method may have a dry matter content about 10% w/w, such as about 15% w/w, about 20% w/w, about 25% w/w, about 30% w/w, about 35% w/w, about 40% w/w, about 45% w/w, about 50% w/w, about 55% w/w, about 60% w/w, about 65% w/w, about 70% w/w, about 75% w/w, about 80% w/w, about 85% w/w or about 90% w/w.

In one embodiment of the inventive method, the waste treatment in step a) can be subjected to water. The dry matter content of the waste fraction can be measured according to Assay VIII. Depending on the dry matter content, water may be added to the waste fraction. For example, when the waste fraction provided is municipal solid waste (MSW) it may be convenient to subject the waste fraction to water in an amount of about 0.5 to about 3.0 kg water per kg MSW. In one embodiment of the invention, the waste fraction may be subjected to about 0.5 to about 2.5 kg water per kg MSW. In a preferred embodiment of the invention, the water fraction may be subjected to about 0.8 to about 1.8 kg water per kg MSW. As a result of adding water to the waste fraction, the dry matter content of the waste fraction including water is lower than the waster fraction before addition of water.

In a preferred embodiment of the invention, the waste fraction is subjected to water to obtain a water to waste ratio in the range of about 0.1:1 to 5:1, preferably in the range of 0.5:1 to 3:1, more preferably in the range of 1:1 to 2:1, even more preferably in the range of 1:1 to 1.5:1.

The method of the present invention comprises subjecting the waste to an enzyme composition in step a). The purpose of the enzyme composition is to treat the biodegradable material present on the waste fraction. The biodegradable material is thereby degraded to smaller fractions, e.g. by enzymes that can hydrolyse carbohydrates to sugar molecules.

Suitable enzyme compositions are well known in the art and are commercially available. A suitable enzyme composition is for instance a composition comprising a cellulolytic background composition (CBC) combined with one or more enzymes.

When added to the process the cellulolytic background composition (CBC) may comprise a commercial cellulolytic enzyme preparation. Examples of commercial cellulolytic enzyme preparations suitable for use in the method according to the present invention include but is not limited to, for example, CELLIC® CTec (Novozymes A/S), CELLIC® CTec2 (Novozymes A/S), CELLIC® CTec3 (Novozymes A/S), CELLUCLAST® (Novozymes A/S), NOVOZYM™ 188 (Novozymes A/S), SPEZYME™ CP (Genencor Int.), ACCELLERASE™ TRIO (DuPont), FILTRASE® NL (DSM); METHAPLUS® S/L 100 (DSM), ROHAMENT™ 7069 W (Rohm GmbH), or ALTERNAFUEL® CMAX3™ (Dyadic International, Inc.).

When the enzyme composition comprises further enzymatic activity apart from the activities present in the CBC, such enzyme activity may be added from individual sources or together as part of enzyme blends. Suitable blends include but are not limited to the commercially available enzyme compositions Cellulase PLUS, Xylanase PLUS, BrewZyme LP, FibreZyme G200 and NCE BG PLUS from Dyadic International (Jupiter, FL, USA) or Optimash BG from Genencor (Rochester, NY, USA).

The CBC may comprise the following enzymatic activities:

Cellobiohydrolase I:

Endo-1,4-beta-glucanase

Beta-glucosidase

Endo-1,4-beta-xylanase

Beta-xylosidase

Beta-L-arabinofuranosidase

Amyloglocosidase

Alpha-amylase

Acetyl xylan esterase

In a preferred embodiment, the activity of the CBC is in accordance with the activity of ACCELLERASE® TRIO™ (Genencor Int.), Cellic CTec2 (Novozymes A/S) or Cellic CTec3 (Novozymes A/S).

The enzyme composition may comprise about 40-99% w/w of an enzyme having cellulolytic activity. In one embodiment, the enzyme composition comprises about 50-90% w/w of an enzyme having cellulolytic activity, such as about 60-80% w/w of an enzyme having cellulolytic activity or about 65-75% w/w of an enzyme having cellulolytic activity. The enzyme composition may comprise about 0-20% w/w of a protease, e.g. about 10% w/w of the enzyme composition. The enzyme composition may comprise about 0-30% w/w of a beta-glucanase, e.g. about 15% w/w of the enzyme composition. The enzyme composition may comprise about 0-10% w/w of a pectate-lyase, e.g. 5% w/w of the enzyme composition. The enzyme composition may comprise about 0-10% w/w of a mannanase or an amylase, e.g. about 5% w/w of the enzyme composition.

The waste may be subjected to the enzyme composition at a concentration of about 10-20 kg enzyme composition per tons of waste, preferably about 12-19 kg enzyme composition per tons of waste, more preferably about 14-17 kg enzyme composition per tons of waste. In a preferred embodiment, the waste may be subjected to the enzyme composition at a concentration of about 16 kg enzyme composition per tons of waste.

The process of the invention comprises in step a) subjecting the waste fraction to an enzyme composition and reacting at a pH between 3.0 and 6.0 and at a temperature of between 40° C. and 60° C. in order to obtain a bioliquid.

In one embodiment of the invention, the pH in step a) is below 6.0, preferably below 5.0, more preferably below 4.5, even more preferably below 4.4 and most preferably below 4.2. The pH may be in the range of 3.0-6.0, such as in the range of 3.0-5.8, such as in the range of 3.5, 4.0-5.5, in the range of 4.0-5.0, in the range of 4.0-4.5 or in the range of 4.0-4.4.

The temperature in step a) of the inventive method is 55° C. or below, the temperature is 50° C. or below or the temperature is 45° C. or below. In one embodiment of the invention, the temperature is in the range of 40-55° C., in the range of 40-50° C. or in the range of 40-45° C.

In one embodiment of the invention the pH in step a) is in the range of 3.0-6.0 and the temperature is in the range of 40-55° C. In a further embodiment, the pH is in the range of 4.0-5.8 and the temperature is in the range of 40-55° C. More preferably the pH is in the range of 4.0-5.5 and the temperature is in the range of 40-50° C. More preferably the pH is in the range of 4.0-5.0 and the temperature is in the range of 40-45° C.

In one embodiment of the invention, the waste is subjected to the enzyme composition for a period of 10-30 hours, preferably 20-25 hours and more preferably about 18 hours.
In a preferred embodiment the invention pertains to a method for sanitizing waste, the method comprising:
   a) Subjecting waste comprising biodegradable material and non-biodegradable material and having a total bacterial count of at least 2.5×10⁸ CFU/gram waste, a bacterial count of E. coli of at least 1.5×10⁶ CFU/gram waste and/or a bacterial count of Enterobacteriaceae of at least 1.5×10⁸ CFU/gram waste, to enzymatic and/or microbial treatment in a bioreactor at a pH between 4.0 and and at a temperature of between 40° C. and 50° C. for a period of 18 to 25 hours to obtain at least partial reduction in bacterial count.
In another preferred embodiment, the invention pertains to a method for sanitizing waste, the method comprising:
   a) Subjecting waste comprising biodegradable material and non-biodegradable material and having a total bacterial count of at least 2.5×10⁸ CFU/gram waste, a bacterial count of E. coli of at least 1.5×10⁶ CFU/gram waste and/or a bacterial count of Enterobacteriaceae of at least 1.5×10⁸ CFU/gram waste, to enzymatic and/or microbial treatment in a bioreactor at a pH between 4.0 and and at a temperature of between 40° C. and 50° C. for a period of 18 to 25 hours to obtain at least partial reduction in bacterial count.
   b) subjecting the treated waste from step a) to one or more separation step(s), whereby a bioliquid and a solid fraction is provided;
   c) subjecting said bioliquid and/or solid fraction to downstream processing

Low temperatures during reaction with enzymes are advantageous as fuel for heating the waste fraction to high temperatures, e.g. 75° C., is saved. Considerable savings are available when waste fractions are reacted with enzymes for about 10 to 30 hours. A further advantage is that the workers handling the inventive method are not exposed to high temperatures.

It was previously believed that waste fractions should be pre-treated at temperatures of 90-95° C. before being used for producing a bioliquid. The effect of the pre-treatment is a sterilization of the waste fraction, whereby undesired microorganism, e.g. pathogenic bacteria, were killed. WO2013/185778 teaches that pre-heating of waste is not necessary. The application shows that by addition of microorganisms (inoculation of EC12B) and enzymes to waste and allowing concurrent enzymatic treatment and microbial fermentation at temperatures of 45-75° C., a safe fermentation can be achieved.

The inventors of the present invention have surprisingly found that when reacting a waste fraction with a specific content of natural occurring bacteria and enzyme at low temperatures (40-60° C.), the resulting bioliquid and non-biodegradable waste material has very low numbers of bacteria recognized as excellent indicator bacteria: Enterobacteriaceae and E. coll. As a result, the bioliquid, the non-biodegradable material and the equipment used do not expose the environment to undesired bacteria, e.g. pathogens. Thus, a safer environment is achieved, especially for the workers handling the inventive method and workers sorting the waste after the waste is separated from the bioliquid.

Various foodborne viruses, blood viruses, and faecal-oral transmitted viruses may also be present in the waste, depending on the waste. However, the process conditions described in step a) and/or step c) of the current invention completely inactivates or reduces the viruses such as e.g. Corona viruses, Adenovirus, Herpes viruses, Measles, HIV, and Flu viruses to a non-harmful level during the processing. In one embodiment of the invention sanitization includes reduction or inactivation of virus.

The process of the invention further comprises a recovery of the bioliquid by separating the bioliquid from the non-biodegradable material. The bioliquid can be separated by one or more separation means such as one or more ballistic separator(s), sieve(s), washing drum(s), presses and/or hydraulic press(es). In one embodiment of the invention, the bioliquid is separated from the waste fraction by use of a ballistic separator.

The one or more separation means separate the bioliquid from the waste. The waste can comprise several types of non-biodegradable materials such as textiles and foils (2D) and cans and plastic bottles (3D).

The water used for rinsing the non-biodegradable waste can be recirculated, heated and subjected to the waste fraction under step a) of the inventive method.

Inert material which is sand, and glass is typically removed e.g. sieved from the bioliquid. Metals are typically removed from all waste fractions. The 2D fraction can further be separated into recyclables and/or residuals such as Solid Recovered Fuel (SRF), Refused Derived Fuel (RDF) and/or inerts. The 3D fraction can also be further separated into recyclables and/or residuals such as metals, 3D plastic and/or RDF.

In one embodiment of the invention, the bioliquid produced by the inventive method is processed into biofuel, e.g. biogas.

According to the Health Protection Agency (Guidelines for assessing the microbiological safety of ready-to-eat foods placed on the marked, Health Protection Agency, London, November 2009, https://webarchive.nationalarchives.gov.uk/20140714111812/http://www.hpa.org.uk/webc/HP AwebFile/HPAweb_C/1259151921557) the amount of Enterobacteriaceae in ready-to-eat food should be below 1×10² CFU/ml in order to be satisfactory. An amount of Enterobacteriaceae of more than 1×10⁴ CFU/ml is unsatisfactory in ready-to-eat food, whereas an amount of 1×10²-1×10⁴ CFU/ml is borderline.

The Health Protection Agency recommend the bacterial count of E. coli to be below 20 CFU/ml in order to be satisfactory for ready-to-eat food. A bacterial count of E. coli in the range of 20-1×10² CFU/ml is borderline and bacterial count of E. coli above 1×10² is unsatisfactory in ready-to-eat food.

In a further aspect, the invention pertains to a bioliquid produced by inventive method. By the inventive method it is possible to produce a bioliquid, which satisfies the microbial requirements to ready-to-eat food products.

In one embodiment of the invention, the bioliquid produced comprises very low number of pathogenic bacteria, e.g. E. coli.

In one embodiment of the invention, the bioliquid has a bacterial count for Enterobacteriaceae below 1×10²-1×10⁴ CFU/ml as measured by Assay I, preferably below 1×10² CFU/ml.

In one embodiment of the invention, the bioliquid has a bacterial count for E. coli below 20-100 CFU/ml as measured by Assay II, preferably below 20 CFU/ml and more preferably below 10 CFU/ml.

In one embodiment of the invention, the bioliquid has a bacterial count for Lactic Acid Bacteria of at least 1×10⁵ CFU/ml as measured by Assay III, preferably at least 1×10⁶ CFU/ml.

The invention further concerns non-biodegradable waste material obtainable from the inventive method. The non-biodegradable waste material can be 2D or 3D material, which may be cleaned after being separated from the bioliquid. In one embodiment of the invention, the non-biodegradable is 2D waste.

In one embodiment of the invention, the non-biodegradable waste material has a bacterial count for Enterobacteriaceae below 1×10²-1×10⁴ CFU/ml as measured by Assay IV, preferably below 1×10² CFU/ml.

In one embodiment of the invention, the non-biodegradable waste material has a bacterial count for E. coli below 20-100 CFU/ml as measured by Assay II, preferably below 20 CFU/ml and more preferably below 10 CFU/ml.

In one embodiment of the invention, the non-biodegradable waste material has a bacterial count for Lactic Acid Bacteria of at least 1×10⁵ CFU/ml as measured by Assay III, preferably at least 1×10⁶ CFU/ml.

In one embodiment of the invention, the ratio between the bacterial count of lactic acid bacteria (CFU/ml) and the total bacteria count (CFU/ml) is at least 1:2 to 1:1.

In one aspect, the invention pertains to biogas produced from the bio liquid obtained by the inventive method.

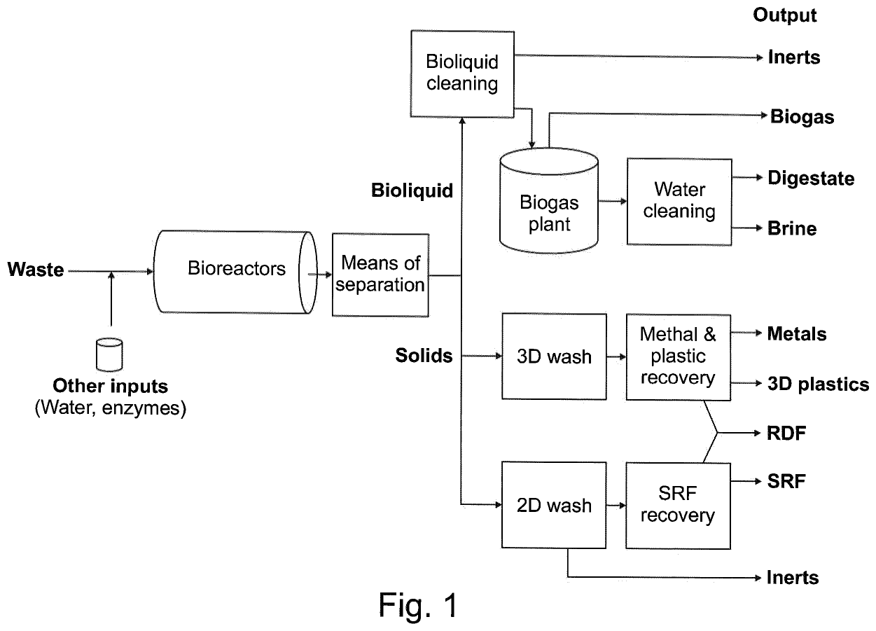

FIG. 1 is a schematic overview of a waste process and is explained in more details below.

During this first stage, means to open plastic bags and adequate pulping or shredding of degradable components are typically provided (not shown in FIG. 1), preparing the waste to be a more homogeneous organic phase before addition of enzymes. In some cases, removal of initial fractions such as metal or other material can take place before the waste is placed in the bioreactor. In some cases, reduction of particle size distribution or upfront sorting of the material is performed. Water, enzymes, and/or microorganisms are added. The enzymatic liquefaction and/or saccharification and/or microbial fermentation is performed continuously at the optimal residence time, temperature and pH for enzyme and microbial performance. Through this enzymatic treatment and fermentation, the biogenic part of the MSW is liquefied and/or saccharified into a bioliquid comprising inter alia mono-, di- and/or oligosaccharides.

The method of the invention may sanitize waste, such as MSW, comprising objects of different size, in one embodiment of the invention the large solid objects are pre-sorted before the waste entrees the bioreactor. The method according to the invention are effective on objects of various particle size. In one embodiment the method according to the invention is applied to objects which have a maximum particle size of 600 mm, such as 500 mm, such as 400 mm, such as 300 mm, such as 200 mm, such as 100 mm, such as 80 mm, such as 70 mm, such as 60 mm or such as 50 mm.

In the separation step, the bioliquid is separated from the non-degradable fractions. The separation is typically performed by one or more separation means such as one or more ballistic separator(s), sieve(s), washing drum(s), presses and/or hydraulic press(es). The bioliquid can be cleaned and then be further processed into biogas in the biogas plant. The one or more separation means separate the waste, such as MSW, treated with enzyme and/or microbial action, into the bioliquid, a fraction of 2D materials, e.g. non-biodegradable materials, and a fraction of 3D materials, e.g., non-biodegradable materials. The 3D fraction (such as cans and plastic bottles) does not bind large amounts of bioliquid, so a single washing step is often enough to clean the 3D fraction. The 2D fraction (textiles and foils as examples) typically binds a significant amount of bioliquid. Therefore, the 2D fraction is typically pressed using e.g. a screw press, washed and pressed again to optimize the recovery of bioliquid and to obtain a cleaner and drier 2D fraction. Inert material which is sand, and glass is typically removed e.g. sieved from the bioliquid. Metals are typically removed from all mentioned fractions. The water used in one or more of the washing drums can be recirculated, heated and then used for heating of the waste during the first step. The 2D fraction can be further separated into recyclables and/or residuals such as SRF (Solid Recovered Fuel), RDF (Refused Derived Fuel) and/or inerts. The 3D fraction can also be further separated into recyclables and/or residuals such as metals, 3D plastic and/or RDF.

### EXAMPLES

Assays

Assay I: Total Bacterial Count

1 ml from each dilution of bioliquid was plated on petrifilm plates “3M™ Petrifilm™ Aerobic Count Plate” for total bacterial count. Petrifilm plates were incubated for 48 hours at 30° C., after which colony forming units (CFU) were counted according to the manufacturer's instructions.

Assay II: Lactic Acid Bacterial Count

1 ml from each dilution of bioliquid was plated on petrifilm plates “3M™ Petrifilm™ Lactic Acid Bacteria Count Plate” for lactic acid bacterial count. Petrifilm plates were incubated for 48 hours at 37° C., after which colony forming units (CFU) were counted according to the manufacturer's instructions.

Assay III: Enterobacteriaceae Count

1 ml from each dilution of bioliquid was plated on petrifilm plates “3M Petrifilm™ Enterobacteriaceae Count Plate for Enterobacteriaceae” count. Petrifilm plates were incubated for 48 hours at 37° C., after which colony forming units (CFU) were counted according to the manufacturer's instructions.

Assay IV: Escherichia Colt Count

1 ml from each dilution of bioliquid was plated on petrifilm plates “3M Petrifilm™ type E. coli and Coliform Count” for Escherichia coli count. Petrifilm plates were incubated for 48 hours at 37° C., after which colony forming units (CFU) were counted according to the manufacturer's instructions.

Assay V: Aerobic Bacteria Count

The total amount of aerobic bacteria count was performed using Yeast Extract Agar (YEA). From each dilution of bioliquid, 1 ml of sample was plated onto an empty petri dish (1 petri dish per sample). Then molten YEA, cooled to approx. 47° C., was poured into the petri dish and mixed with the sample so there would be equal distribution of bacterial growth within the agar. Once the agar was set, the plates were then incubated at 30° C. for 72 hours after which CFU were counted.

Assay VI: Enterobacteriaceae Count

Enterobacteriaceae count was performed using Violet Red Bile Glucose Agar (VRBGA). From each dilution of bioliquid, 1 ml of sample was plated onto an empty petri dish (1 petri dish per sample). Then molten VRBGA, cooled to approx. 47° C., was poured into the petri dish and mixed with the sample so there would be equal distribution of bacterial growth within the agar. Once the agar was set an overlay of VRBGA was added too and the plates were then incubated at 37° C. for 24 hours after which CFU were counted.

Assay VII: E. coli Count

E. coli count was performed using Violet Red Bile Agar (VRBA). From each dilution of bioliquid, 1 ml of sample was plated onto an empty petri dish (1 petri dish per sample). Then molten VRBA, cooled to approx. 47° C., was poured into the petri dish and mixed with the sample so there would be equal distribution of bacterial growth within the agar. Once the agar was set an overlay of VRBA was added too and the plates were then incubated at 44° C. for 24 hours after which CFU were counted. All counted colonies had to undergo a confirmation process using MacConkey agar, YEA agar, an oxidase test, Lactose Peptone Water, and Tryptone Water (with Kovacs reagent).

Assay VIII: Dry Matter Content

The dry matter content of a waste can be determined by drying a sample at 60° C. for 48 hours. The weight of the sample before and after drying should be measured and can be used to calculate the dry matter content in percent by the following formula:

Sample weight after drying×100=% dry matter in sample Sample weight before drying

### Example 1

This example investigates the bacterial count of sorted output samples from the method according to the invention and compare this to the bacterial count of output samples from an MBT (Mechanical Biological Treatment) plant. The RDF (Refused Derived Fuel) fraction and the metal from the treatment according to the process of the invention was compared with RDF and metal obtained at an MBT facility in England.

The waste (MSW) subjected to the method according to the present invention had a dry matter content of 50-70%. The MSW was then transported into a bioreactor. Water was added to the MSW to obtain a slurry of waste and water having water to MSW ratio of 1.5-2 to 1. Cellic CTec3® (Novozymes A/S) in a concentration of 0.9-2.3% w/w (based on the weight of the MSW before addition of water) enzyme composition was added to the MSW slurry, which was then allowed to react for 24 hours at a temperature of between 40° C. and 60° C., a pH between 4.0 and 6.0.

The waste entering the MBT plant had a dry matter content of 50-70%. The MSW was sorted into RDF, metal and biodegradable material, before the biodegradable material was transported into a bioreactor.

RDF from both the process of the invention and the MBT facility was sampled and analyzed as follows: 5 individual and separate samples, obtained from various sites within an RDF outputs container were pooled and 1 g mixed with 9 ml sterile 0,9% NaCl. The mixture was vortexed and inverting for 1 minute creating dilution 10-1. The RDF sample was hereafter serial diluted 10⁸ times using sterile 0.9% NaCl. 1 ml from each dilution were plated on petrifilm plates according to Assay I, II, Ill and IV described above.

Metal from the process of the invention and the MBT facility, was sampled and analyzed as follows:
A lid from a standard can (containing e.g. tuna or baked beans) with a size of −77 cm 2 was swabbed with 5 sterile swab sticks, followed by the sticks being placed in 1 ml of appropriate media. The 5 ml were then pooled, and serial diluted using sterile 0.9% NaCl H₂O to 10⁻⁸. 1 ml from each dilution was plated on petrifilm plates according to Assay I, II, Ill and IV described above.

Bacterial counts on a metal can lid obtained from the treatment method according to the invention (test 1) and on a metal can lid obtained from the MBT (test 2), respectively was compared. Test 3 and 4 investigate the bacterial counts on an RDF obtained from the treatment method according to the invention and on an RDF obtained from MBT, respectively. The results are shown in table 1 and discussed below.

TABLE 1

| | | | Total | Lactic | | |
|---|---|---|---|---|---|---|
| | | | bacterial | acid | | |
| | Temperature | | count | bacteria | Enterobacteriaceae | E. coli |
| Test | ° C. | pH | (Assay I) | (Assay II) | (Assay III) | (Assay IV) |
| 1 | 39-48 | 4.0- | 3.5 × 10⁵ | 1.78 × 10⁵ | 5.11 × 10² | <10 |
| Invention | | 4.5 | CFU/lid | CFU/lid | CFU/lid | CFU/lid |
| metal | | | | | | |
| 2 | 39-48 | 4.0- | 2.21 × 10⁷ | 1.25 × 10⁵ | 4.1 × 10⁵ | 2.1 × 10⁴ |
| MBT | | 4.5 | CFU/lid | CFU/lid | CFU/lid | CFU/lid |
| metal | | | | | | |
| 3 | 39-48 | 4.0- | 2.59 × 10⁷ | 3.37 × 10⁶ | 4.88 × 10² | 0 |
| Invention | | 4.5 | CFU/g | CFU/g | CFU/g RDF | CFU/g RDF |
| RDF | | | RDF | RDF | | |
| 4 | 39-48 | 4.0- | 7.46 × 10⁷ | 1.31 × 10⁷ | 3.48 × 10⁵ | 2.94 × 10⁴ |
| MBT | | 4.5 | CFU/g | CFU/g | CFU/g RDF | CFU/g RDF |
| RDF | | | RDF | RDF | | |

Test 1

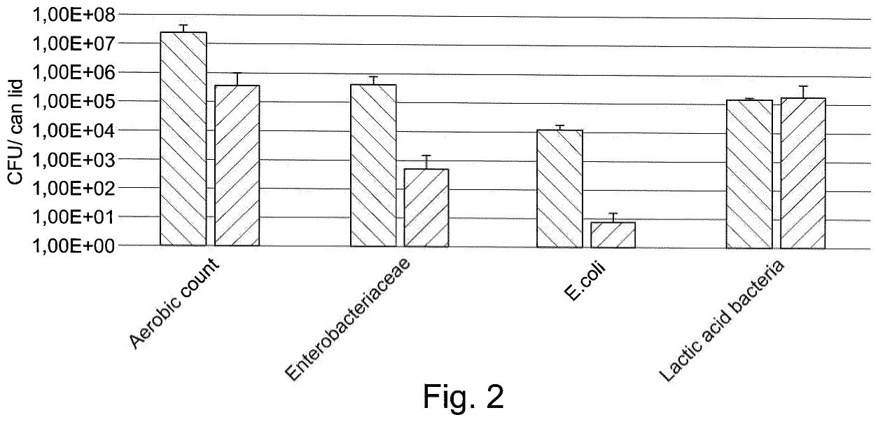

On average the total amount of bacteria was 3.5×10⁵ CFU/can lid, while the pathogenic indicator bacterial count was: Enterobacteriaceae count 5.11×10² CFU/can lid comprising about 1/700 of the entire live bacterial population and E. coli count 6.6×10⁰ CFU/can lid comprising about 1/5000 of the entire live bacterial population. Lactic acid bacterial count was 1.78×10⁵ CFU//can lid and therefore comprised about ½ of the entire live bacterial population in sorted metal samples of the method according to the present invention (FIG. 2). The amount of the indicator bacteria Enterobacteriaceae and E. coli were surprisingly low in the sorted metal treated according to the process of the invention.

Test 2

On average the total amount of bacteria was 2.21×10⁷ CFU/can lid, while the pathogenic indicator bacterial count was: Enterobacteriaceae count 4.10×10⁵ CFU/can lid comprising about 1/54 of the entire live bacterial population and E. coli count 2.1×10⁴ CFU/can lid comprising about 1/1000 of the entire live bacterial population. Lactic acid bacterial count was 1.25×10⁵ CFU/can lid and therefore comprised about 1/175 of the entire live bacterial population in MBT sorted metal (FIG. 2). Thus, the amount of the indicator bacteria Enterobacteriaceae is about 4 times higher than lactic acid bacteria in MBT sorted metal.

Comparison of Test 1 and Test 2

The number of bacteria were compared between MBT sorted metal (Test 1) and sorted metal treated by the process of the invention (Test 2). In MBT sorted metal the total amount of bacteria was >60 times higher compared to sorted metal derived from the process of the invention (FIG. 2). When compared, the Enterobacteriaceae count of MBT sorted metal was >800 times higher than invention sorted metal and the E. coli count >1800 times higher in MBT sorted metal than in invention sorted metal (FIG. 2). Lastly, the lactic acid bacterial were similar between the MBT sorted metal and the invention sorted metal.

These findings suggest two things 1) Better growth conditions for bacteria, including pathogenic indicator bacteria (Enterobacteriaceae and E. cob), but excluding lactic acid bacteria in MBT sorted metal compared to invention sorted metal and 2) The conditions in the bioreactor using the method according to the invention creates a unique environment capable of annihilating pathogenic bacteria.

Test 3

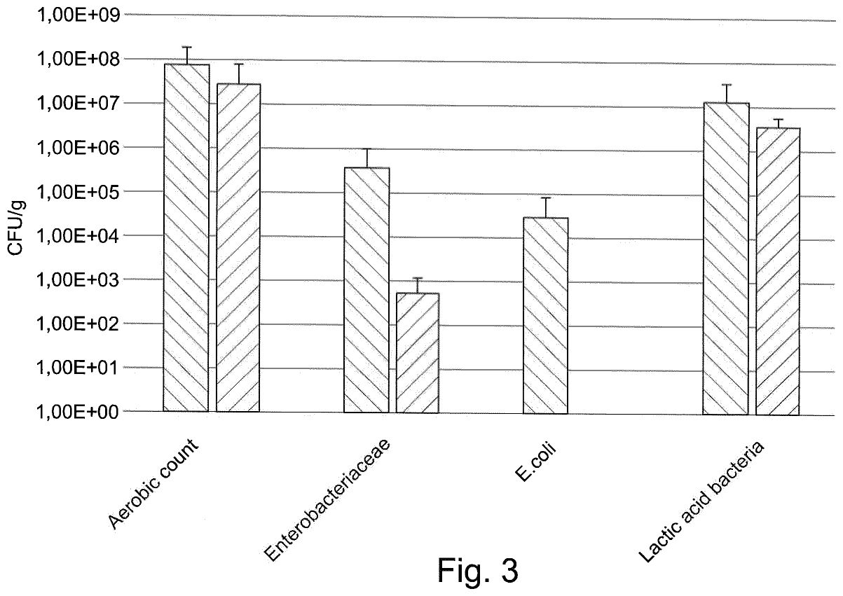

On average the total amount of bacteria was 2.59×10⁷ CFU/g RDF, while the pathogenic indicator bacterial count was: Enterobacteriaceae count 4.88×10² CFU/g RDF and E. coli count 0 CFU/g RDF. Lactic acid bacterial count was 3.37×10⁶ CFU/g RDF and therefore comprised about 1/7 of the entire live bacterial population in invention sorted RDF (FIG. 3). The amount of pathogenic indicator bacterial group Enterobacteriaceae and E. coli were surprisingly low in the sorted invention RDF.

Test 4

On average the total amount of bacteria was 7.46×10⁷ CFU/g RDF, while the pathogenic indicator bacterial count was: Enterobacteriaceae count 3.48×10⁵ CFU/g RDF comprising about 1/138 of the entire live bacterial population and E. coli count 2.94×10⁴ CFU/g RDF comprising about 1/1500 of the entire live bacterial population. Lactic acid bacterial count was 1.31×10⁷ CFU/g RDF and therefore comprised about 1/7 of the entire live bacterial population in MBT sorted RDF (FIG. 3).

Comparison of Bacterial Counts in MBT Sorted RDF and RDF Obtained from the Process of the Invention

The number of bacteria were compared between MBT sorted RDF (test 3) and sorted RDF obtained from a process of the invention (test 4). In MBT sorted RDF total amount of bacteria was >2 times higher compared to sorted RDF obtained from a process of the invention (FIG. 3). When compared, the Enterobacteriaceae count of MBT sorted RDF was >700 times higher than sorted RDF obtained from a process of the invention and the E. coli count >29000 times higher in MBT sorted RDF than in sorted RDF obtained from a process of the invention (FIG. 3). Lastly, the lactic acid bacteria count was >3 times higher in MBT sorted RDF compared to sorted RDF obtained from a process of the invention. These findings suggest two things 1) Better growth conditions for bacteria, including pathogenic indicator bacteria (Enterobacteriaceae and E. coli) in MBT sorted RDF, compared to sorted RDF obtained from a process of the invention and 2) The conditions in the bioreactor using the method according to the invention creates a unique environment capable of annihilating pathogenic bacteria.

### Example 2—pH and Temperature Tests

In order to establish specific boundaries (ranges) in regard to pH and temperature in which pathogenic bacteria are exterminated in the method according to the process of the invention, experiments using model waste (model MSW) were carried out.

Model MSW was utilized to mimic MSW as described below.

“Model MSW” can be prepared in order to mimic the composition of real municipal solid waste. The below describes the composition of model MSW consisting of 3 fractions:
41% vegetable fraction (cf. Table 2)
13% protein/fat fraction (animal origin) (cf. Table 3) and
46% cellulosic fraction (cf. Table 4).

TABLE 2
Vegetable fraction of model MSW

| Composition of | % of vegetable |
|---|---|
| model MSW | fraction (weight %) |
| Onions | 7.5 |
| Carrots | 7.5 |
| Potatoes | 6.3 |
| Leeks | 4.4 |
| Salad | 3.2 |
| Frozen peas | 4.4 |
| Tomatoes | 3.2 |
| Cucumber | 3.2 |
| Red cabbage | 3.2 |
| Mushrooms | 3.2 |
| Oatmeal | 3.2 |
| Cornflakes | 4.4 |
| Apples, bananas, oranges, | 4.4 |
| lemons, pears | |
| Remoulade | 3.2 |
| Ketchup | 3.2 |
| Rye bread | 6.3 |
| White bread | 9.5 |
| Cake | 3.2 |
| Flowers | 1 |
| Coffee grounds | 1 |
| Boiled rice | 3 |
| Boiled pasta | 3 |
| Celery | 3 |
| Brussels sprout, kale | 3.5 |
| Beans, lentils | 1 |
| Broccoli | 0.25 |
| Cauliflower | 0.25 |
| Green beans | 0.25 |
| Pineapple | 0.15 |

TABLE 3
Protein/fat fraction (animal origin) of model MSW

| Composition of | % of protein/fat fraction |
|---|---|
| model MSW | (animal origin) (weight %) |
| Roasted pork | 6 |
| Dog/cat food | 6 |
| Liver paté | 5 |
| Salami | 5 |
| Mortadella | 5 |
| Liver sausage | 5 |
| Ham | 5 |
| Rolled sausage | 5 |
| Hotwings | 10 |
| Spareribs | 5.5 |
| Fat of animal origin with spices | 10 |
| Cheese | 4 |
| Ymer (soured whole milk) | 10 |
| Eggs | 3 |
| Shrimps | 3 |
| Herring | 5 |
| Ground beef | 1.5 |
| Chicken whole | 2 |
| Chicken filet | 4 |

TABLE 4
Cellulose fraction of model MSW

| Composition of | % of cellulose fraction |
|---|---|
| model MSW | (weight %) |
| Milk cartons | 30.0 |
| Newspapers | 8.0 |
| Magazines | 2.8 |
| Advertising materials | 9.7 |
| Phone books | 0.7 |
| Printing paper | 2.2 |
| Gift wrapping | 6.2 |
| Cardboard | 9.8 |
| Paper towel | 22.5 |
| Cotton pads | 1.7 |
| Wood | 1.2 |
| Textiles (dishtowels) | 5.3 |

Fermentations were carried out under the conditions set out in Table 5.

TABLE 5

| Experiment | pH | Temperature |
|---|---|---|
| 1 | 6 | 50 |
| 2 | 4 | 20 |
| 3 | 4 | 50 |
| 4 | 6 | 20 |
| 5 | 5 | 35 |
| 6 | 5 | 35 |
| 7 | 5 | 35 |
| 8 | 6 | 20 |
| 9 | 4 | 20 |
| 10 | 4 | 50 |
| 11 | 6 | 50 |
| 12 | 6 | 25 |
| 13 | 5.5 | 30 |
| 14 | 6 | 35 |
| 15 | 5 | 25 |
| 16 | 4.5 | 25 |

Fermentations were performed in Sartorius™ 1 L equipped with mechanical stirrer, heating mantle, cooling mantle, cooling tower for exhaust gases and pH-meter. The temperature was varied (see table 5) using an electrical heating or cooling mantle and the stirring was 600 rpm. pH was adjusted to appropriate values by addition of 1M HCl or 1M NaOH through the Sartorius™ automated pumping system. The added components (solids and liquids) were not pre-heated prior to addition into the fermenter.

Fermentation of model MSW was carried out using 166 g model MSW, 1 L de-ionized water and 4 g Cellic® CTec3 (Novozymes A/S). First water and model MSW was heated or cooled to appropriate temperature (see table 5) while stirring (300 rpm). Simultaneously, pH was adjusted to appropriate (see table 5) setting by addition of HCl or NaOH. Upon reaching the desired temperature and pH, Cellic Ctec3® was added (4 g) and the stirring increased to 600 rpm. This was followed by the addition of 1.3 ml Escherichia colt (strain DSM 498) in an approximate concentration of 8×10⁸ CFU/ml.

E. coli was grown overnight in nutrient broth at 37° C. shaking overnight prior to addition to fermenters. Furthermore, E. coli overnight culture, was centrifuged for 5 min, 5000 rpm and the pellet resuspend in 0,9% NaCl H₂O to an OD₆₀₀=1.

Sample Acquisition and Analysis

Samples of about 10 mL were withdrawn from the fermenters and the resulting Bioliquid was serial diluted using sterile 0.9% NaCl H₂O to 10⁻⁸. 1 ml from each dilution was plated on petrifilm plates. The bacterial count of the indicator bacteria Enterobacteriaceae and E. coli were measured according to Assays III and IV.

Indicator bacteria are counted in order to validate a specific environment for growth capabilities of pathogenic bacteria. The Enterobacteriaceae group (such as E. cols) are living in the mammal intestine as commensals, with the ability to become pathogenic. E. coli has been recognized as excellent indicator bacteria for decades. If these organisms are found to be present in an environment, this could indicate that pathogenic bacteria in general is capable of growth in that particular environment.

TABLE 6
Measured E. coli counts for various pH and temperatures

| | Temper- | | | CFU | −log₁₀(CFU |
|---|---|---|---|---|---|
| | ature | | | (24 h)/ | (24 h)/ |
| pH | ° C. | 0 Hours | 24 Hours | CFU (0 h) | CFU (0 h)) |
| 6 | 50 | 1.24E+06 | 8.40E+03 | 6.03E−03 | 2.220 |
| 4 | 20 | 1.17E+06 | 0.00E+00 | 0 | 9.908* |
| 4 | 50 | 1.57E+06 | 0.00E+00 | 0 | 9.959* |
| 6 | 20 | 2.77E+06 | 1.63E+05 | 5.31E−02 | 1.275 |
| 5 | 35 | 1.58E+06 | 4.60E+02 | 3.44E−04 | 3.464 |
| 5 | 35 | 7.30E+05 | 3.50E+02 | 6.00E−04 | 3.222 |
| 5 | 35 | 7.40E+05 | 1.30E+02 | 1.67E−04 | 3.778 |
| 6 | 20 | 5.50E+05 | 3.40E+03 | 7.22E−03 | 2.141 |
| 4 | 20 | 1.40E+06 | 8.00E+01 | 6.34E−05 | 4.198 |
| 4 | 50 | 8.55E+05 | 0.00E+00 | 0 | 9.803* |
| 6 | 50 | 1.29E+06 | 6.00E+02 | 3.16E−04 | 3.501 |
| 6 | 25 | 1.38E+06 | 8.20E+03 | 5.91E−03 | 2.228 |
| 5.5 | 30 | 1.60E+06 | 1.27E+04 | 7.37E−03 | 2.133 |
| 6 | 35 | 1.41E+06 | 9.65E+05 | 6.53E−01 | 0.185 |
| 5 | 25 | 1.35E+06 | 5.00E+03 | 3.83E−03 | 2.417 |
| 4.5 | 25 | 1.25E+06 | 6.00E+01 | 5.73E−05 | 4.242 |

*to apply the logarithmic transformation to the relative CFU counts, the values of 0 were replaced with 10⁻⁴

The obtained experimental data was analysed in Design-Expert software, version 11 (Stat-Ease, Inc.). To model the obtained results the following transformations were applied to the data:
1. The ratio of CFU counts after 24 h and at the beginning of the experiment was calculated
2. If the ratio from point 1 equals to 0, the ratio was substituted with 10-4 to be able to apply a logarithmic transformation to all the ratios.
3. The negative logarithm with base 10 of the ratios was calculated.
4. To make the data more normally distributed, a power transformation with power of 0.59 was applied to the values from point 3.
5. The final model was built including the pH, temperature (T) and squared temperature (T²) terms. The model was shown to be significant (p<0.0001), pH (p<0.0001) and T² (p=0.0341) terms were significant as well. T term (p=0.0635) was included because of the T² term. The lack of fit was not significant (p=0.6876). R² for the model is 0.84, predicted R² is 0.695.

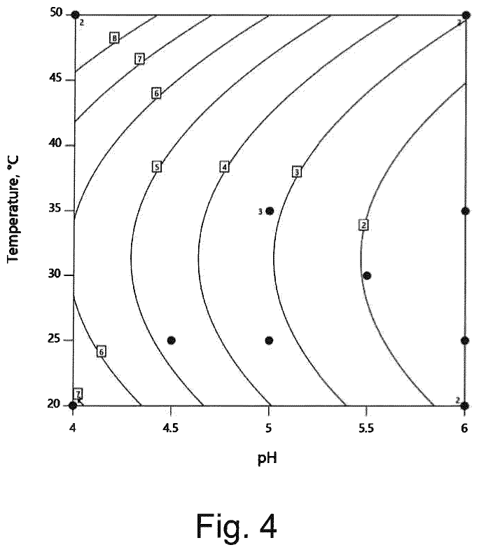

The predicted boundaries for non-pathogenic bacterial growth in regard to pH and temperature while running the method according to the invention are depicted in FIG. 4. The lines represent a 10 log relative reduction of E. coli by that temperature and pH. i.e. line 2 equals 10 log 2=100 relative E. coli CFU reduction. line 5 equals 10 log 5=100.000 relative E. coli CFU reduction and so forth. Thus. pathogenic E. coli are reduced by a 10 log 5 on the left side of line 5. Dots represent experiments carried out.

### Example 3: Lab Scale Liquefaction of Model Waste without Previous Hygienization

A series of separate fermentations were carried out using 166 gram model MSW (prepared as described in Example 2), 1 Liter de-ionized water and either 2, 4, 6 or 8 gram of Cellic® Ctec3 (purchased from Novozymes A/S) in which a fixed amount of inoculum, derived from Foulum biogas plant in Denmark, from the before-mentioned CSTR digester were added (166 gram). First water, inoculum (166 gram) and model MSW was heated to 50° C. while stirring (300 rpm) in a 5 L Sartorius™ fermenter. Upon reaching the desired temperature, enzyme (Cellic CTec3™) was added (2, 4, 6 or 8 grams) and the stirring increased to 1200 rpm for 5 minutes and thereafter to 900 rpm for 1 hour. After 1 hour of stirring, the stirring was reduced to 600 rpm until the end of the experiment. The concentration of glucose, xylose, lactate, acetate and ethanol was measured at time points 18.25, 25.50, 42.50, 47.50 and 114 h after addition of enzyme by HPLC.

TABLE 7
Data obtained using 2 gram Cellic Ctec3 (all in g/L)

| Time (h) | Glucose | Xylose | Lactate | Acetate | Ethanol | pH |
|---|---|---|---|---|---|---|
| 18.25 | 3.5 | 1.0 | 0.5 | 0.5 | 3.4 | 5.40 |
| 25.50 | 3.3 | 0.9 | 0.8 | 0.5 | 4.2 | 5.24 |
| 42.50 | 4.1 | 1.4 | 3.7 | 0.7 | 6.3 | 4.94 |
| 47.50 | 3.3 | 1.4 | 5.0 | 0.7 | 6.5 | 4.83 |
| 114 | 3.0 | 1.7 | 7.4 | 0.8 | 4.4 | 4.61 |

TABLE 8
Data obtained using 4 gram Cellic Ctec3 (all in g/L)

| Time (h) | Glucose | Xylose | Lactate | Acetate | Ethanol | pH |
|---|---|---|---|---|---|---|
| 18.25 | 7.0 | 1.8 | 1.6 | 0.8 | 2.4 | 5.07 |
| 25.50 | 7.3 | 1.7 | 1.8 | 0.7 | 3.5 | 4.98 |
| 42.50 | 5.3 | 1.7 | 7.7 | 1.0 | 3.9 | 4.39 |
| 47.50 | 5.5 | 1.6 | 7.3 | 0.8 | 4.5 | 4.27 |
| 114 | 5.6 | 1.9 | 8.9 | 1.0 | 3.0 | 4.15 |

TABLE 9
Data obtained using 6 gram Cellic Ctec3 (all in g/L)

| Time (h) | Glucose | Xylose | Lactate | Acetate | Ethanol | pH |
|---|---|---|---|---|---|---|
| 18.25 | 5.4 | 1.3 | 1.0 | 0.5 | 1.7 | 5.03 |
| 25.50 | 6.0 | 1.4 | 1.6 | 0.6 | 2.7 | 4.92 |
| 42.50 | 6.2 | 1.6 | 6.3 | 0.9 | 4.5 | 4.22 |
| 47.50 | 4.8 | 1.7 | 8.3 | 0.9 | 3.8 | 4.13 |
| 114 | 5.7 | 1.9 | 8.8 | 1.1 | 2.6 | 4.09 |

TABLE 10
Data obtained using 8 gram Cellic Ctec3 (all in g/L)

| Time (h) | Glucose | Xylose | Lactate | Acetate | Ethanol | pH |
|---|---|---|---|---|---|---|
| 18.25 | 6.8 | 2.1 | 3.5 | 0.7 | 0.3 | 4.80 |
| 25.50 | 7.6 | 2.4 | 8.4 | 1.0 | 0.4 | 4.28 |
| 42.50 | 7.2 | 2.1 | 9.9 | 0.9 | 0.4 | 4.12 |
| 47.50 | 7.2 | 2.1 | 9.8 | 1.0 | 0.0 | 4.16 |
| 114 | 8.3 | 2.2 | 9.1 | 1.2 | 0.3 | 4.36 |

In all four experiments, model MSW was solubilized and glucose was released. The methanogenic bacteria from the inoculum resulted in a significant ethanol production in the experiment with low enzyme loading. Consequently, there was less glucose available for the lactic acid bacteria and the acidification was much slower with pH staying above 5 for about hours. When the enzyme dose was increased there was a gradually faster acidification and with the high enzyme dose (8 g) the pH dropped below 4.5 within 24 hours. This also effectively limits the production of ethanol and acetate to 0.3 and 1.2 g/L, respectively. These experiments show that hygienization of the reject water may be beneficial if the inherent lactic acid producing community in the waste is limited whereas hygienization of the reject water is not necessary if a sufficient large lactic acid community is present in the waste because the lactic acid bacteria is able to outcompete the inherent methanogenic bacteria present in the reject water.

1. Method for sanitizing waste, the method comprising:
a) Subjecting waste comprising biodegradable material and non-biodegradable material and having a total bacterial count of at least 2.5×10⁸ CFU/gram waste, a bacterial count of E. coli of at least 1.5×10⁶ CFU/gram waste or a bacterial count of Enterobacteriaceae of at least 1.5×10⁸ CFU/gram waste, to enzymatic and/or microbial treatment in a bioreactor at a pH between 3.0 and 6.0 and at a temperature of between 40° C. and 60° C. for a period of 10 to 30 hours to obtain at least partial reduction in bacterial count.

2. Method according to claim 1 further comprising:
b) subjecting the treated waste from step a) to one or more separation step(s), whereby a bioliquid and a solid fraction is provided;
c) subjecting said bioliquid and/or solid fraction to downstream processing.

3. Method according to any of the preceding claims, wherein the waste has a dry-matter content in in the range of 50-70% wt.

4. Method according to any of the preceding claims, wherein the waste under step a) is subjected to water to reduce the dry matter content of the waste.

5. Method according to any of the preceding claims, wherein the waste is subjected to enzymatic and/or microbial treatment in a bioreactor for a period of about 20-25 hours, preferably about 18 hours.

6. Method according to any of the preceding claims, wherein the pH in step a) is below 6.0, preferably below 5.0, more preferably below 4.5, even more preferably below 4.4 and most preferably below 4.2.

7. Method according to any of the preceding claims, wherein the temperature in step a) is 55° C. or below, the temperature is 50° C. or below or the temperature is 45° C. or below.

8. Method according to any of the preceding claims, wherein the waste is subjected to enzymatic and/or microbial treatment in a bioreactor in step a) for 24 hours and the pH is 4-6 and the temperature is in the range of 40-60° C.

9. Bioliquid obtainable by the method of claims 1-8.

10. The bioliquid according to claim 9, where the bioliquid has a bacterial count for Enterobacteriaceae below 1×10 2-1×10 4 CFU/ml as measured by Assay III, preferably below 1×10² CFU/ml and/or, where the bioliquid has a bacterial count for E. coli below 20-100 CFU/ml as measured by Assay IV, preferably below 20 CFU/ml and more preferably below 10 CFU/ml.

11. The bioliquid according to any of claims 9-10, where the bioliquid has a bacterial count for Lactic Acid Bacteria of at least 1×10⁵ CFU/ml as measured by Assay II, preferably at least 1×10⁶ CFU/ml.

12. Non-biodegradable waste material obtainable from the method of claims 1-8.

13. The non-biodegradable waste material according to claim 12, where the material has a bacterial count for Enterobacteriaceae below 1×10²-1×10⁴ CFU/ml as measured by Assay IV, preferably below 1×10² CFU/ml.

14. The non-biodegradable waste material according to any of claims 12-13, where the material has a bacterial count for E. coli below 20-100 CFU/ml as measured by Assay II, preferably below 20 CFU/ml and more preferably below 10 CFU/ml.

15. The non-biodegradable waste material according to any of claims 12-14, where the material has a bacterial count for Lactic Acid Bacteria of at least 1×10⁵ CFU/ml as measured by Assay III, preferably at least 1×10⁶ CFU/ml.
